(19) United States
(12) Patent Application Publication
(10) Pub. No.: US 2022/0111004 A1
(43) Pub. Date: Apr. 14, 2022

(54) Combinatorial Apparatus for Disease Management

(71) Applicant: David M Manyak, Burgess, VA (US)

(72) Inventor: David M Manyak, Burgess, VA (US)

(21) Appl. No.: 17/345,531

(22) Filed: Jun. 11, 2021

Related U.S. Application Data
(63) Continuation of application No. 17/332,683, filed on May 27, 2021.
(60) Provisional application No. 63/038,265, filed on Jun. 12, 2020.

(51) Int. Cl. A61K 38/17; A61K 45/06; C12Q 1/6827

(52) U.S. Cl. CPC A61K 38/1732; C12Q 1/6827; A61K 45/06

(57) ABSTRACT

This disclosure provides for the application of a multi-disciplinary analysis of information sources to draw novel conclusions that result in new methods to diagnose, prevent or treat COVID-19. COVID-19 appears to be an extremely complex disease, encompassing three critical aspects at least: a viral infection, an immune system disorder, and a cardiovascular/pulmonary/renal disease with significant coagulation system dysregulation. This disclosure principally focuses on the design and methods of use of a combinatorial apparatus that addresses critical needs to treat patients with COVID-19, especially those at high risk of, or experiencing, adverse effects of COVID-19 infection, including but not limited to kidney function support, supplemental oxygen administration, correction of cardiovascular dysfunction, and removal or modification of deleterious molecules or agents from or in a patient's blood, including virus particles or molecular components thereof. Applications of the combinatorial device for disease management other than for use with respect to COVID-19 are also described.

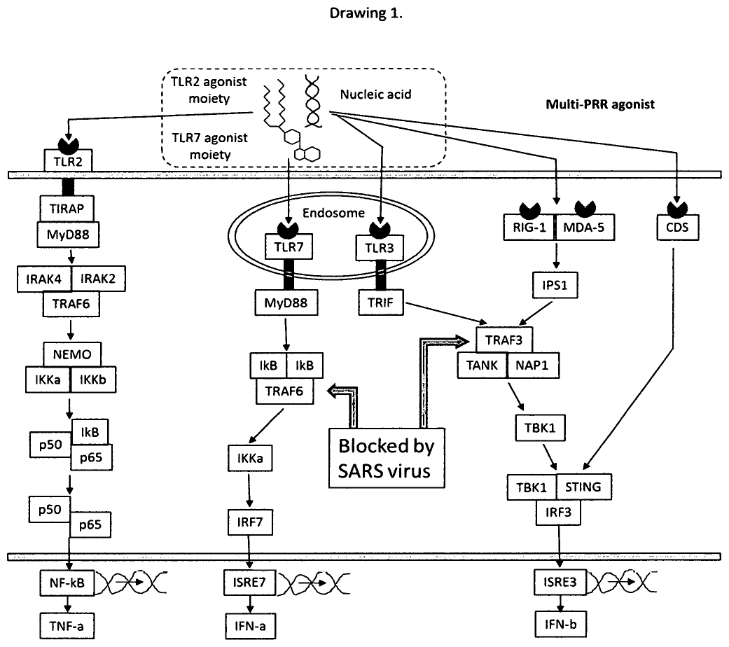

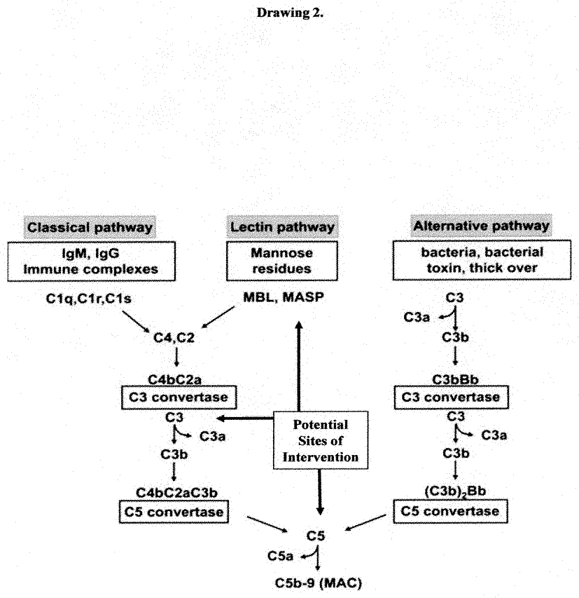

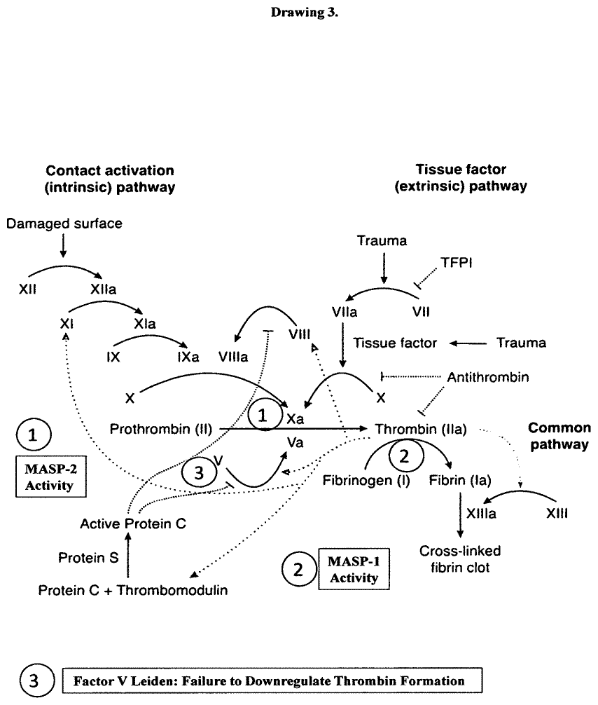

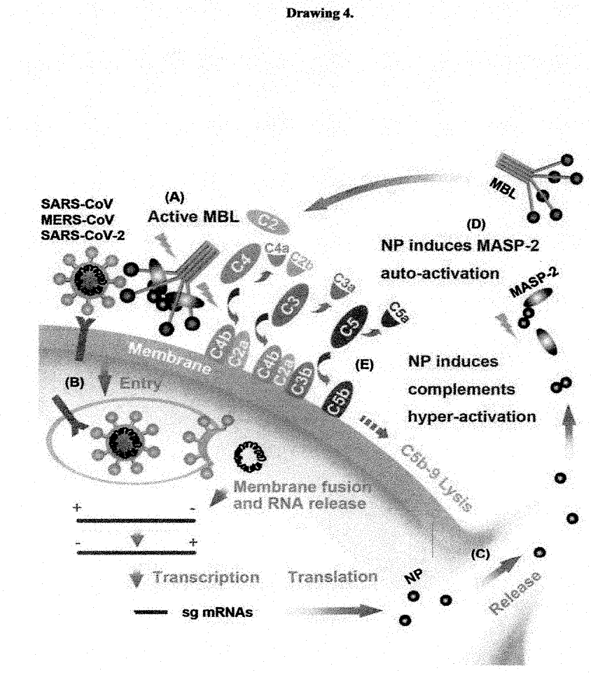

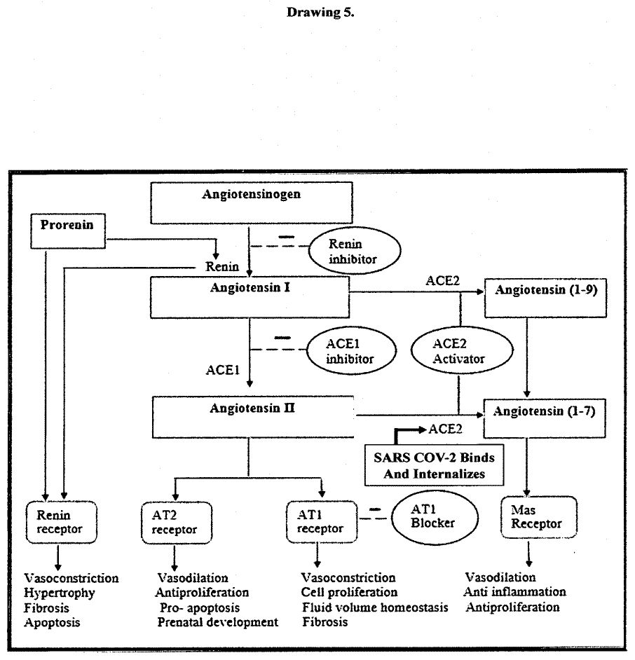

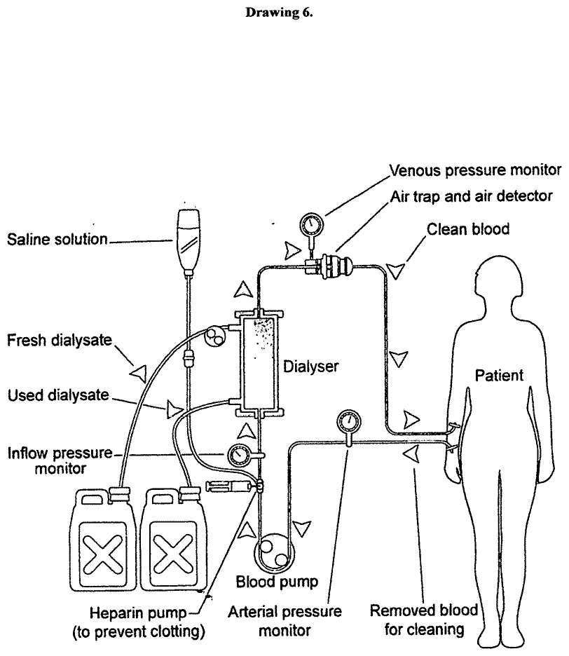

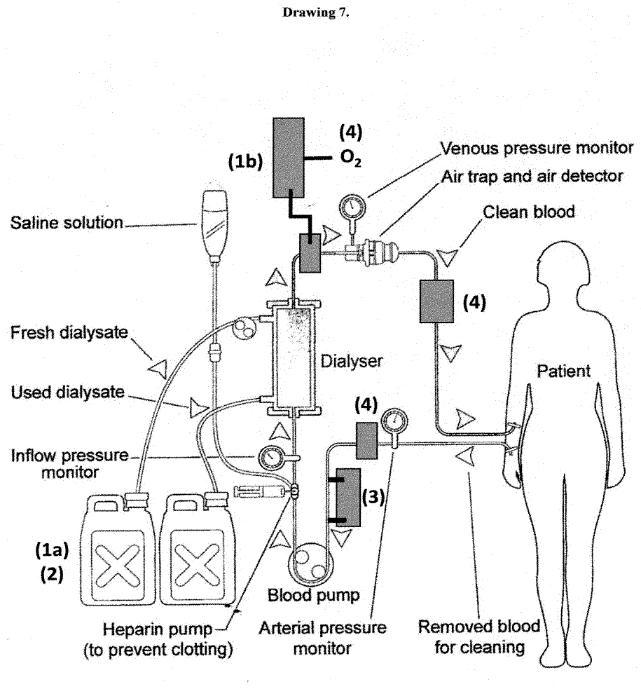

### CROSS REFERENCE TO RELATED APPLICATIONS. (RELATED APPLICATIONS MAY BE LISTED ON AN APPLICATION DATA SHEET, EITHER INSTEAD OF OR TOGETHER WITH BEING LISTED IN THE SPECIFICATION.)

This application claims the benefit of U.S. Provisional Application Ser. No. 63/038,265, filed Jun. 12, 2020 and U.S. Nonprovisional application Ser. No. 17/332,683 filed May 27, 2021. The entire disclosures of the provisional and nonprovisional applications are relied upon and incorporated by reference herein.

### STATEMENT OF FEDERALLY SPONSORED RESEARCH OR DEVELOPMENT (IF ANY)

None.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

Not Applicable.

### REFERENCE TO A “SEQUENCE LISTING,” A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC. THE TOTAL NUMBER OF COMPACT DISC INCLUDING DUPLICATES AND THE FILES ON EACH COMPACT DISC SHALL BE SPECIFIED

Not Applicable.

### BACKGROUND OF THE INVENTION

The COVID-19 Pandemic

In late 2019, a widespread outbreak of viral infections initially centered in and around Wuhan, China, was identified. By March 2020, the outbreak had spread to a worldwide pandemic, affecting nearly every country and causing high morbidity and mortality. The infectious agent has been identified as a beta coronavirus (a single-stranded RNA virus), termed SARS COV-2, which is a novel infectious agent to which no general immunity had been established in human populations, and for which no available vaccine nor any known treatment for the resultant disease, termed COVID-19, existed. Therefore, there is a critical need for new unforeseen methods of diagnosis, prevention, mitigation, treatment, and cure to be discovered, developed, and applied. With the rapid rate of infection, time is of the essence for these critical needs.

Human coronaviruses were discovered in the 1960's. Relatively mild strains of coronavirus are thought to account for about a quarter of cases of the common cold. More deadly forms of coronavirus infections of humans emerged in the 2000's, notably SARS (2002-2004), which like COVID-19 was initially centered in China, and MERS (2012), centered in the Middle East. SARS in particular resulted in more than 8000 cases worldwide, with an approximately 10% mortality rate. A significant amount of scientific research was conducted related to SARS, but with limited follow-up to therapies or preventatives, in part because the disease spread stopped. No new cases of SARS have been reported since 2004. Nevertheless, the SARS research was important since the SARS coronavirus and SARS COV-2 share many similarities in the viral strains' genomes, biology, and modes of infection and virulence.

Although the general perception is that COVID-19 should be treated as a viral infection, and in particular a respiratory infection, many of the symptoms and morbidities exhibited by COVID-19 suggest a much broader range of effects than a simple respiratory infection. In fact, COVID-19 appears to be an extremely complex disease, encompassing three critical aspects at least: a viral infection, an immune system disorder, and a cardiovascular/pulmonary/renal disease with significant coagulation system dysregulation. Moreover, it may be that these aspects of COVID-19 are at least in part sequential, with the possibility that the viral “respiratory” infection is resolved relatively quickly in many patients (neutralizing antibodies and viral clearance are being reported as early as a week after symptoms of infection appear) while the adverse effects from dysfunction of the cardiovascular/pulmonary/renal and coagulation systems rage on in severe cases over many weeks. The latter are therefore important aspects of COVID-19 for therapeutic intervention.

The SARS COV-2 Virus is Reported to Attack through the ACE2 Receptor

From a putative bat source and/or an intermediate animal host, coronaviruses have jumped to infect humans. For SARS and MERS, the person-to-person infectivity seemed to be relatively modest. Once the initial infection cycle ran its course, with the infected individuals building immunity and person-to-person transmission stopped, the SARS and MERS outbreaks ended. As noted, there have been no documented cases of SARS since 2004. With COVID-19, however, the person-to-person or potentially even surface-to-person transmission is much stronger. Even so, so far research has suggested that the mechanisms of infection are highly similar between SARS and SARS COV-2, but with the SARS COV-2 virus gaining a mutation or slightly modified genetic adaptation for enhanced functionality at the key virus binding and entry site on host cells, another set of mutations or genetic inserts that enhance the cleavage of a surface protein, or spike protein, of the SARS CoV-2 virus facilitating entry into host cells, and potentially gaining a greater evasive advantage with respect to the human immune defense system. Clearly these differences make SARS COV-2 more highly infectious and cause more far-ranging effects on human health vs. SARS.

The coronavirus that caused the SARS outbreak in 2002-04 has been shown to specifically bind to, and gain entry into human cells through internalization by, Angiotensin Converting Enzyme-2 (ACE2), a protease receptor found on the surface of many cell types. Recent studies indicate that SARS COV-2 infects human cells through the same ACE2 target as the SARS coronavirus. These coronaviruses contain external knobs (Spike proteins, which are specifically glycosylated) that can bind to the ACE2 receptor with high affinity. Once the SARS or SARS COV-2 virus binds to the ACE2 receptor, a second step is required for entry of the virus into the host cell. A protease, on the host cell surface, at least one of which has been identified as the trypsin-like serine protease called Transmembrane Serine Protease 2 (TMPRSS2), or perhaps a protease present in circulation or associated with a different cell type, cleaves a section of viral spike protein, which allows the remaining section of the spike protein to mediate fusion of the virus envelope membrane with the host cell membrane.

On SARS COV-2 Spike protein, there are two cleavage sites associated with this activation and fusion. One, the S1/S2 cleavage site, sometimes referred to as a “furin site”, has an insert of additional genetic sequences on SARS COV-2 compared with SARS. Based on modeling studies, this insert allows the site at which the cleaving serine protease (e.g., TMPRSS2, or furin) attaches and cuts to bulge out in a loop from the Spike protein. This loop, carrying a positive charge from two new arginine residues, presumably would facilitate attraction of a binding pocket of a serine protease with an embedded negative charge, such as an aspartic acid residue, increasing the effectiveness or probability of cleavage at the S1/S2 site. The result would be an increased host membrane fusing activity and therefore a more highly infectious SARS COV-2 virus. The second site, the S2 cleavage site, is also cut by a serine protease (again, such as TMPRSS2 as well), but appears to be basically the same structural sequence as the SARS virus S2 site.

Once these sites are cleaved, the virus is internalized into the host cell and can take over the machinery of that cell to replicate and make more viral copies. It does this using additional proteins that are part of the virus. Inside the cell, two large polyproteins are formed by translation of the viral RNA by the host cell's own synthetic organelles, and these polyproteins in turn are processed into the non-structural replicating proteins by two SARS COV-2 proteases, called 3C-like proteinase (3CLpro) and papain-like proteinase (PLpro). 3CLpro and PLpro are considered primary targets for protease-inhibitor antiviral drugs against SARS COV-2. Another protein that is formed is a replicase or polymerase used by the virus to make additional copies of RNA for packaging into new virus particles. This replicase is another target for antiviral drug development. Once more copies of the virus are made, the new viral particles are released into the bloodstream to infect additional cells. This process of release involves membrane fusion again between a vesicle inside the cell containing the new virus particles and the host cell membrane. Once released, the additional viral particles can infect other cells by the same or similar process. In addition, separate SARS COV-2 structural proteins such as nucleocapsid (N) protein may be released into the bloodstream.

The sequence of binding SARS CoV-2 to the ACE2 receptor and activation of the viral surface protein to allow cell fusion is not yet definitively known. One primary working hypothesis is that the virus binds to ACE2, then the activation of the viral fusion process occurs through the action of TMPRSS2 that is co-located with ACE2. Or can the virus be pre-activated for fusion somewhere else, then find an ACE2 receptor to bind to and start cell entry without further modification? Or are there alternative activating proteases other than TMPRSS2 and/or are there other molecular targets to which the SARS COV-2 virus can bind or be bound, either allowing entry into cells not expressing ACE2 or allowing proximity to the alternative activating proteases for pre-activation? These are important questions to help define the process of infection after exposure to the SARS COV-2 virus.

### BRIEF SUMMARY OF THE INVENTION

COVID-19, resulting from infection by the coronavirus SARS COV-2, is a multifaceted disease for which there is a critical need for new unforeseen methods of diagnosis, prevention, mitigation, treatment, and cure to be discovered, developed, and applied. COVID-19 appears to be an extremely complex disease, encompassing three critical aspects at least: a viral infection, an immune system disorder, and a cardiovascular/pulmonary/renal disease with significant coagulation system dysregulation. This invention teaches the design and methods of use of a device, or combinatorial apparatus, for management of certain aspects of SARS COV-2 infection and related COVID-19 adverse effects or health issues. The combinatorial apparatus described herein has further utility as a means of treating a disease, infection, adverse event, drug or vaccine side effect, or other medical condition in addition to those related to COVID-19.

### BRIEF DESCRIPTION OF THE DRAWINGS

Drawing 1.

Diagram of signaling pathways for innate immune response mediated by Toll-like Receptor-7 (TLR7) and RIG-1, showing components of pathways (TRAF3 and TRAF6) at which SARS-2 virus can block further signaling by which type 1 interferons would otherwise be stimulated to be expressed as an antiviral response to infection.

Drawing 2.

Diagram of Complement Pathways, from Noris and Remuzzi, (2013), especially the components of the Lectin complement pathway, showing potential sites of therapeutic intervention for treatment of COVID-19 by blocking the pathway at MBL/MASP (-1 and -2) (the preferred components to block) or Complement C3 or Complement C5.

Drawing 3.

Diagram of the Intrinsic and Extrinsic Coagulation pathways, showing sites (enzyme activity) at which (1) MASP-2 can promote the conversion of Prothrombin to Thrombin and (2) MASP-1 can promote the conversion of Fibrinogen to Fibrin, both of which can lead to formation of a fibrin clot, as well as (3) the site at which the polymorphism Factor V Leiden can lead to failure to downregulate the clot formation process by preventing Activated Protein C from converting Factor V to Factor Va, which in turn would otherwise limit further conversion of Prothrombin to Thrombin.

Drawing 4.

Diagram from Gao, T. et al. preprint 2020, “Highly pathogenic coronavirus N protein aggravates lung injury by MASP-2-mediated complement over-activation”, showing potential role of MBL and MASP-2 in potentiating SARS COV-2 replication via a positive feedback loop, and further suggesting use of inhibitors of MBL and/or MASP-2 for treating COVID-19.

Drawing 5.

Diagram of the components and interrelationships between components of the renin-angiotensin system. Angiotensin Converting Enzyme 2 (ACE2), located on the surface of numerous cell types in the cardiovascular system, functions to convert Angiotensin II to Angiotensin 1-7, resulting in vasodilation. SARS COV-2, through its Spike protein, binds to ACE2, and the ACE2/SARS COV-2 complex becomes internalized into the host cell. The resulting reduction in available cell surface ACE2 counteracts the normal vasodilatory activity of ACE2, leading to enhanced vasoconstriction.

Drawing 6.

Diagram of a basic hemodialysis system, with key components and processes (from Wikipedia, https://en.wikipedia.org/wiki/Hemodialysis#/media/File:Hemodialysis-en.svg). The system includes a connection such as a catheter to a human patient's blood system, tubing that allows a patient's blood to flow or be pumped out of the patient into a dialyser chamber, dialysis tubing inside of the dialyser chamber in which dialysate fluid flows countercurrent to the patient's blood flow, additional tubing allowing the patient's blood to flow from the dialyser chamber back to the patient via a connection such as a catheter to the patient's blood system, in-line pressure monitors, and other desirable parts, potentially including but not limited to a heparin injector port to prevent clotting on the inflow side and an air trap and air detector on the outflow side to prevent air bubbles in returning blood.

Drawing 7.

Diagram of a Combinatorial Apparatus comprising a component (A) of substantially the same design as a basic hemodialysis system as shown in Drawing 6, or a modification thereof, plus a component (B) that provides supplemental oxygen to the patient's blood flowing through the apparatus as shown for example by increasing oxygen saturation in the dialysate (Ia) or the addition of an oxygen injection system (1b) and optionally one or more oxygen sensors or measurement devices for other parameters (4) that feeds back to control the oxygen injection device, or to control other functions of the combinatorial apparatus, plus a component (C) comprising a molecular capture module (3) for binding and removing from the blood macromolecules or particles such as viruses or viral components or for enzymatically modifying substances in a patient's blood. The combinatorial apparatus also allows for disease intervention and management by the addition of small molecules to the dialysate (2) to achieve a desired concentration of the small molecule in the patient's blood.

### DETAILED DESCRIPTION OF THE INVENTION

### Summary of the Invention

COVID-19, resulting from infection by the coronavirus SARS COV-2, is a multifaceted disease for which there is a critical need for new unforeseen methods of diagnosis, prevention, mitigation, treatment, and cure to be discovered, developed, and applied. This invention relates to a multi-disciplinary analysis of information sources to draw novel conclusions about methods to diagnose, prevent or treat COVID-19.

This invention teaches that interferon alpha or interferon beta be administered to high risk individuals prophylactically and to early diagnosed patients with COVID-19, based on the analysis that bats, which are natural reservoirs for coronaviruses but show no ill effects, unlike humans express type 1 interferon constitutively, and on the analysis that SARS COV-2 can inhibit the production of beneficial antiviral type 1 interferons in humans.

This invention teaches that lectins, especially those that bind mannose and/or N-acetylglucosamine, and in particular lectins such as banana lectin and griffithsin and derivatives thereof, are useful for diagnostic methods, prevention, and treatment of SARS COV-2 infection because of their high binding affinity for glycosylated proteins on the surfaces of viruses similar to those on the spike proteins of SARS COV-2 and their demonstrated ability to limit viral infection through such binding. Specific uses and methods are detailed in the Description below.

This invention teaches that, for SARS COV-2 that enters the gastrointestinal tract and potentially infects an individual through the intestines, consumption of a mannose lectin, preferably banana lectin in banana pulp, on a schedule and an amount sufficient to bind SARS COV-2 in a way to limit or prevent the virus' systemic entry through the intestines, is a potential treatment for COVID-19. Such consumption of banana may also allow banana lectin to enter the bloodstream through the intestines and bind to SARS COV-2 in such a way to limit infection by the virus into an individual's cells as an additional treatment modality for COVID-19.

This invention teaches that SARS COV-2 enters the gastrointestinal tract, that the small intestines are a site of potential systemic infection, and that some proportion of the virus will pass through the digestive system into feces, so that diagnostic tests based on fecal samples should be implemented and handling guidelines for items exposed to feces especially in high risk settings such as nursing homes need to be established for epidemiological control purposes. This invention also teaches that infectious SARS COV-2 is likely to be present in semen based on analysis of target and activating molecules in the testes and prostate, and that guidelines for abstaining from unprotected sex while individuals are infected should be established for epidemiological control purposes.

This invention teaches that the lectin complement pathway, including mannose-binding lectin (MBL) and its associated serine proteases, MASP-1 and MASP-2, play a central role in COVID-19, both positively in their innate immune system function and adversely, especially in severe cases in which the coagulation system becomes dysregulated and/or red blood cells are destroyed. This invention teaches that administration of inhibitors or blockers of MASP-1 and/or MASP-2 after the onset of infection is a preferred method of treating these adverse symptoms. This invention further teaches that inhibitors or blockers of components further down in the complement cascade such as complement C3 or complement C5 are also potential treatments for these adverse effects.

This invention teaches that dysregulation of the coagulation system evidenced in children and young adults with COVID-19, exhibiting symptoms being described as a Kawasaki Disease-like condition, is likely associated with genetic polymorphisms in one or more components of the coagulation cascade. In particular, individuals with Factor V Leiden mutation may be especially vulnerable to these adverse coagulation events, and genetic testing for Factor V Leiden should be implemented as a means to identify these at risk patients. This invention further teaches that inhibitors or blockers of MASP-1 and/or MASP-2 are potential treatments for this Kawasaki Disease-like syndrome associated with COVID-19.

This invention teaches that susceptibility to SARS COV-2 infection may be enhanced in individuals who have polymorphisms in the mbl2 gene that result in low or undetectable levels of the MBL protein. This invention teaches that an early diagnostic test following SARS COV-2 confirmation should be to measure circulating MBL levels in blood for evidence of depressed readings. This invention further teaches that screening for mbl2 genetic polymorphisms should be implemented to identify individuals with a heightened risk of SARS COV-2 infection.

This invention teaches the administration of inhibitors or blockers of trypsin-like serine proteases as a means of treating or preventing COVID-19 by limiting or preventing activation of the SARS COV-2 virus. Specific blockers may be plant-based serine protease inhibitors such as sunflower protease inhibitor (SFPI-1) that are administered orally including by consumption as a food. Another specific blocker may be an inhibitor or blocker of MASP-1 or MASP-2 administered systemically.

This invention teaches that the interaction of SARS COV-2 with its ACE2 cellular target results in adverse cardiovascular complications in COVID-19 patients, especially in the lungs and kidneys which are sites of high levels of expressed ACE2, and that such adverse effects include vasoconstriction and likely blockage of blood flow through capillaries.

This invention teaches that certain gene mutations may predispose individuals to more severe adverse consequences of vasoconstriction on blood flow through capillary beds, such as the mutation resulting in sickle cell disease which is more prevalent in individuals of Sub-Saharan African descent than in Caucasians. Screening for sickle trait in COVID-19 patients may help with management of the disease.

This invention teaches that administration of angiotensin 1-7 or agonists of the Mas receptor, including AVE0991 or CGEN-856S, may be beneficial for treating vasoconstriction caused by SARS COV-2 binding to the ACE2 receptor.

This invention further provides for a novel combinatorial apparatus for the regular, as needed, hemodialysis treatments for pre-existing kidney disease patients while undergoing hospitalization for COVID-19, especially those patients who need to be provided with supplemental oxygen that would otherwise be provided by mechanical ventilators, and renal function support for non-pre-existing dialysis patients who experience or may experience kidney failure or kidney malfunction as a result of SARS COV-2 infection.

This invention teaches the deployment of a combinatorial apparatus and methods that would provide a superior option for administering supplemental oxygen and other therapeutic support to COVID-19 patients in general, compared with the currently used mechanical ventilators, by administering the supplemental oxygen directly into the bloodstream rather than through the lungs. Certain adverse effects on the cardiovascular and coagulation systems may make ventilators relatively ineffective in re-oxygenating the blood of severe COVID-19 patients.

This invention teaches providing therapeutic support in the form of supplementing the dialysate used in the combinatorial apparatus with disease-specific or symptom-specific molecules, such as Ang 1-7, that are able to diffuse through the dialysate tubing in the system into the patient's blood, and/or to remove deleterious or excess diffusible molecules from the blood while passaging through the apparatus.

This invention further teaches an aspect of therapeutic support by including a capture module in the combinatorial apparatus that would enable larger molecules or other substances or agents, including viral particles such as SARS COV-2, that cannot pass through the dialysis tubing, to be bound to an immobilized capture substrate in the module and removed from the patient's blood stream. Alternatively, reactive molecules such as enzymes, including ACE2, could be immobilized to surfaces in the capture module, in which case the reactive molecules would act upon molecules in the patient's blood to alter, inactivate, or otherwise modify them as part of a therapeutic regimen.

Descriptions of the combinatorial apparatus use therapeutic intervention in COVID-19 patients as examples for use and utility of the combinatorial apparatus. It should be clear however that the combinatorial apparatus described herein, or components thereof, could be widely used for management of other diseases. Advantages of this combinatorial apparatus would include (1) the ability to administer a drug or other small molecule (that is, one diffusible through the selected dialysis tubing molecular weight cut-off) to a patient to rapidly and continuously achieve a steady state concentration of that drug or other small molecule in the blood or serum, (2) the ability to manage the desired concentration of small molecules in the blood stream by adding them or deleting them, or increasing or decreasing their concentration, in the dialysate fluid such that by countercurrent flow of the dialysate and patient blood a desired concentration of the targeted small molecule is achieved in the blood being returned to the patient's body, (3) the ability to remove larger molecules or infectious agents from the patient's blood through exposure to an immobilized capture molecule or substance through contact between the patient's blood and the exposed surface containing the immobilized molecule, without having to directly administer (e.g., by intravenous injection) the capture molecule into the patient, thus potentially eliminating or reducing potential side effects of having the capture agent (such as an antibody) circulating freely in the bloodstream, and (4) the ability to alter molecules in the patient's bloodstream through their interaction with an immobilized molecule (such as an enzyme) or other material within the capture module, again thus potentially eliminating or reducing potential side effects of having the modifying agent (such as an enzyme) circulating freely in the bloodstream if administered directly into the patient. In each of these cases, if any adverse effects of the therapeutic interventions are seen, that intervention can be immediately terminated by altering the composition of the dialysate fluid or by bypassing the patient's blood flow so it does not flow through the capture module. Other uses of this combinatorial apparatus should be readily apparent to those ordinarily skilled in the art.

Other inventions should be apparent from the Descriptions presented below.

### DESCRIPTION OF THE INVENTIONS

The Tissue Distribution of ACE2 Receptor Suggests Potential Sites of Entry and Infection

Given the above reported mechanism, it is important to understand where ACE2 is expressed as a protein in the body, in not yet infected individuals, and specifically where it is expressed at a site where there is potential exposure to the external environment, allowing transmission and infection from the external environment to the individual. These sites are most likely the sites of transmission of the virus from one person to another, or from viruses on surfaces into a person. According to a study from 2004 at the time of the SARS outbreak using more traditional histology methods (Hamming et al., 2004), the ACE2 protein, as one would expect from it being a key component of blood pressure regulation systems, is expressed on the epithelial cells lining blood vessels throughout the body, including within most organs. These sites, however, are all in general (except for tissue tears or open wounds) not exposed to the external environment. The two organs with extensive expression on surfaces affronting the external environment were the lungs (Type I and Type 2 alveolar cells) and the small intestines where ACE2 expression is concentrated in the brush border of the epithelial cell layer lining the small intestines, where the body takes up food nutrients. Therefore, these are two of the most likely sites of entry by the virus. Both the lungs and digestive system (e.g., small intestines) interface with the environment though the mouth and nasopharyngeal space. Importantly, Hamming et al. found high expression of ACE2 in the nasopharyngeal space but only on the basal (internal to the body) side, below the thick layer of squamous cells lining the mouth and nasopharyngeal space, suggesting that the mouth and throat may not be primary sites of viral attachment and entry into the body.

More recently (Ziegler et al., 2020), using single cell expression analysis, reported ACE2 RNA transcript expression in human nasal secretory (goblet) cells and ciliated cells in the mucosa of the upper airway and nasopharyngeal space. While these cells could represent additional and perhaps initial sites of infection by SARS COV-2, the percentages of such cells expressing ACE2 were relatively low (1-4% of these cell types). Furthermore, an even lower percentage of these cells (0.3%-1.6%) expressed both ACE2 plus the presumed activating enzyme TMPRSS2.

While these cell types may well be initial sites of infection, their low density raises questions about whether this entry point would be sufficient to account for a widespread systemic infection. These cells, however, may represent the key sites at which components of the innate immune system in the mucosa (e.g., toll-like receptors) recognize and mount an initial response against the virus. Thus, low titers of virus exposure (but above a threshold required for a response) may induce an antiviral response at this level leading to asymptomatic cases of COVID-19 and eventual protective immunity. In this regard, Ziegler et al. (2020) also reported wide expression of genes that upregulate alpha interferon in these same cell types expressing ACE2.

Therefore, the most likely sites of infection are the small intestines, the oral/nasopharyngeal area, and the lungs. Most of the public advisories for COVID-19 prevention have focused on preventing transmission via airways (wearing masks, avoiding close exposure to aerosols from other individuals, etc.), yet ACE2 expression, based on classical histology, appears to be found in low levels in the upper respiratory tract, and the most likely movement of viruses that enter an individual through aerosol into the upper airway would tend to be back up into the oral/nasal area. The mouth, throat, nasal passages and upper airway tract are lined with the mucosa, a layer of mucus secreted by the endothelial cells that entraps particles that enter the nasopharyngeal space. Furthermore, these regions have extensive cilia on the endothelial cells that beat in a way to move the mucus up out of the airways and out of the nasal passages into the throat. There the mucus (with entrapped viral particles) is either expectorated or ingested down into the stomach. Similarly, saliva secreted into the mouth, which might also entrap or bind viruses, also is either spit out or passed down into the digestive tract.

Coupled with the fact that the worst areas of infections of the lungs with COVID-19 are deep in the lungs, not necessarily in the upper respiratory region, the above description would imply that a primary site of entry of, and systemic infection by, SARS COV-2 might be in the digestive system, not just the respiratory system, and rather that the lower lobes of the lungs may be infected in large part after the virus enters the body. In this case, SARS COV-2 would enter via ACE2 expressed on the Type II and Type I alveolar cells through circulation in the blood after primary infection elsewhere (although high levels of more deeply inhaled virus, or sustained inhalation or aspiration of virus particles over a longer period of time, could potentially enter deeper into the airways and directly infect the alveolar cells). This implication of a substantial role of infection through the digestive tract is a surprising conclusion given that the public responses to control of COVID-19 have been based on the assumption that it is a traditional respiratory infection. And if correct this analysis has a number of potential important implications for diagnostic testing, epidemiological control, and treatment of COVID-19, as discussed below.

The ACE2 expressed in the small intestines is densely located on the brush borders of the endothelial cells lining the small intestines. This location provides a ready interface to the external environment for viral infection. In addition to its primary function of regulating key functions in the renin-angiotensin system, ACE2 reportedly has a secondary function of transport of amino acids into the cell. Amino acid absorption from digested food is a primary function of the cells lining the small intestines. Thus, the presence of ACE2 in this cell layer could cause the virus to attach and be internalized, perhaps with activation by TMPRSS2, which is heavily co-expressed with ACE2 in these absorptive enterocytes, and subsequent membrane fusion, or perhaps even with the coincident function of food absorption. Furthermore, under some circumstances normally associated with inflammation, the small intestines exhibit a “leaky gut” syndrome, allowing larger molecules or complexes to leak into the bloodstream. These conditions seem amenable for SARS COV-2 to infect individuals through the small intestines.

Like the oral/nasal region, the surface of the small intestines has an extensive mucosal layer. Non-food particles are trapped in this mucus and carried down the remainder of the digestive tract to be excreted as feces. This may well include virus particles that have not been able to gain entry to infect the individual in the small intestines. Such a phenomenon would imply that SARS COV-2 might be excreted in feces. This has important implications for diagnostic testing and epidemiological control. Such a phenomenon also suggests a potential prophylactic or therapeutic strategy to reduce viral titers infecting the individuals at the time of exposure or soon thereafter.

The following are potential important implications of the above analysis on viral entry sites:
1. To date, the focus of diagnostic testing for SARS COV-2 infection has been on taking nasopharyngeal swabs to use for PCR-based viral RNA detection. If SARS COV-2 is only transiently present in the oral/nasal/upper airway region where the swab is taken because there is not a significant active infection (i.e., not a significant source of ACE2 exposed to the surfaces in this area), then one might expect to have false negatives for detecting infection from this approach, and/or cases where some tests in the same individual are positive and some are negative at different times with the same individual. The same mix of false negatives may occur simply from the scattered distribution of the relatively few goblet and ciliary cells expressing ACE2 at which the virus could infect vs. the location from which the swap is taken. At the least, it might suggest that this diagnostic method is not a reliable early detection method. After a systemic infection occurs, then the oral/nasal/airway may be more highly infected from the infection of endothelial cells in this area that contain ACE2 on the basal side (i.e., toward the bloodstream), in which case the test would more likely detect the presence of virus as these cells become more infected.
2. The likelihood that virus passes through the digestive tract suggests that a diagnostic test using stool samples would be extremely important to deploy, and it is surprising that this approach has not seemed to be part of the public response to COVID-19. Tests for SARS COV-2 using stool samples have been reported in some research settings and have detected SARS COV-2 virus, or at least viral RNA. Whether live virus is present in the samples has apparently not yet been demonstrated, but it is likely. In one study of 131 patients in China (Chen, 2020), positive PCR tests for SARS COV-2 were found in 22 patients in fecal and sputum samples for a period of up to 13 and 39 days respectively after the patient's tests with nasopharyngeal swabs had turned negative. In a case study of an Australian COVID-19 patient, the initial positive (on hospitalization) nasopharyngeal test was followed the next day with a positive fecal test. It takes approximately 4-6 hours for food to reach and pass through the small intestines after ingestion, and 24-44 hours for food remnants to be excreted. So this sampling approach would not be an immediate indication of viral infection, but it may be more reliable if the testing occurred every day especially for high risk individuals such as health care workers or residents of nursing homes. Such testing may provide an indication of exposure to the virus with a 1-2 day lag.
3. Most surprising is that precautions regarding disinfection of surfaces exposed to feces has not been part of the public advisories for preventing infectious spread, if nothing more than a precaution just in case such spread could occur. In particular, one might wonder if the severe spread seen in outbreaks in nursing homes, health care settings, and prisons might be due at least in part to inadequate handling of bedpans, beddings, toilet facilities, and other possible reservoirs for fecal material.
4. Another source for diagnostic testing could be saliva, especially if the sampling is done over a period of time to allow a more widely distributed sample of the oral/nasopharyngeal space and a higher extent of capture of virus to enable a more accurate test. Testing based on sputum could also be a good indicator of deeper lung or upper airway infection but may not be a good test for early diagnosis of SARS COV-2 infection. A potential diagnostic test for systemic or more severe SARS COV-2 infection may be based on use of blood samples, although this testing method would not capture the earliest stages of infection. To date, surprisingly, such a test has apparently not been deployed. A blood-based test would most likely correlate with severity of infection and be important to monitor the rate of clearance of SARS COV-2 infection and the efficacy of antiviral treatments. The current paradigm of nasopharyngeal-based tests, being more scattered or transient in their detection of virus as noted, is unlikely to be useful for such later stage infection monitoring.
5. A diagnostic test based on a virus capture method in the mouth and/or nose, or capture on a mask worn over the mouth and nose, could be the best option for detecting early exposure. In this approach, a much longer exposure time to the virus could be achieved compared with a nasal swab, maximizing the chance of capturing enough viral particles for accurate diagnoses. One possibility for an oral test may be use of a chewing gum or other generally accepted as safe substrate that contains a moiety that binds the SARS COV-2 virus, such as a lectin, preferably a mannose-binding lectin such as banana lectin or Griffithsin or similar molecules, or antibodies against SARS COV-2 surface proteins. In this case, chewing the gum or other substrate for a length of time, perhaps a couple hours, might capture enough virus particles to enable an accurate diagnostic test. This method of detection would also preferably rely on a means to release the captured virus from the substrate after removal from the mouth during the laboratory measurement step. There are numerous examples among products in the life sciences industry in which a capture molecule is bound to a substrate, such as beads in a column or a reactive surface, through a linker to a capture molecule to perform, for example, affinity chromatography. [A representative example is ThermoFisher product 20395, an agarose bead for column chromatography containing bound jacalin (a galactose targeting plant lectin) used to capture secretory IgA. Jacalin could be replaced by a mannose-binding lectin for SARS COV-2, or tested for its ability to bind SARS COV-2.] The captured molecule is then eluted off of the bead or surface by, for example, dissolving or breaking the linker substance. Once eluted and concentrated, a PCR-based or ELISA-type assay could be performed. Another possibility might be the use of chewing tobacco as the oral capture method in which the tobacco plant has been genetically engineered to produce a capture molecule. For example, recombinant Griffithsin, an algae-derived mannose binding lectin with strong affinity for SARS virus, has been manufactured using field-grown tobacco plants (Fuqua et al., 2015, Alam et al., 2018).
6. Biomarker tests for indicators of early infection by SARS-COV-2, rather than detection of SARS COV-2 itself, could also be valuable tools for relatively early detection of COVID-19.

Another site of high levels of expressed ACE2 receptor is the testes. ACE2 is also expressed in the kidney, brain, and heart, or on endothelial cells of blood vessels in these organs, but none of these sites are exposed to the external environment. Nevertheless, once an individual is infected, and the virus enters the blood system, these are all sites for potential cellular infection and adverse effects from alteration to the normal function of the infected cells.

Of particular concern that has not been widely described is the presence of ACE2 expression in the testes, coupled with the known presence of TMPRSS2, as noted an enzyme that is reported to be needed to activate and prime SARS COV-2 for cell fusion and infection, in the prostate. A report from China (Li et al., 2020) indicated that of 38 hospitalized male COVID-19 patients enrolled in a study, 6 had positive diagnoses by PCR in samples of their semen, 2 of whom were in the recovery stage of their infection. TMPRSS2 has been implicated as a marker for prostate cancer, and is reportedly upregulated in response to male hormones (androgens) (Lin et al., 1999). Its normal role in the prostate appears to be processing semen by protease action to make it more fluid. The presence of both ACE2 and TMPRSS2 in the male reproductive organs should raise concerns about whether activated SARS Cov-2 can be sexually transmitted. This could be another important epidemiological control of refraining from unprotected sexual activity while actively infected. Furthermore, this additional reservoir for viral infections, and reported upregulation of TMPRSS2 in response to androgens, may contribute to the observed greater incidence of severe COVID-19 infection in males vs. females.

SARS and SARS COV-2 Coronaviruses Likely Came from Bats

The SARS and SARS COV-2 coronaviruses are thought to be zoonotic (animal-derived), existing in animal reservoirs and being passed to humans from these animals, causing human infections. The putative primary hosts of these two coronaviruses are bats. Bat species have traditionally been divided into megabats (or fruit bats) and microbats (mostly echo-locating insect-eaters), but more recently they have been reclassified (in part on genomic data) into the suborders Yangochiroptera and Yinpterochiroptera, the latter of which include the fruit bats and some of the microbats, including the horseshoe bats. Bats have been shown to be major carriers of a range of viral infections that have affected humans and animals, including important food animals. For example, fruit bats are believed to be the source of the Marburg filovirus outbreak in 2007 and to be a reservoir for Ebola virus. Both certain fruit bats and the related microbats carry Nipah and Hendra viruses. The MERS virus is believed to be from bats, transmitted through camels. The putative source of the SARS coronavirus is a horseshoe bat native to China, possibly transmitted from the bats to humans via a civet cat. And the SARS COV-2 virus is also thought to be derived from bats, possibly through pangolins or snakes as intermediate hosts. This origin for SARS COV-2 is still not definitively known, and genomic analysis has suggested some significant additions or changes in their RNA sequence (i.e., the significant insert at the S1/S2 cleavage site, or furin site) compared with other known coronaviruses, raising questions of an artificially introduced genetic component, or a genetic recombination with another source at some point in time. Nevertheless, bats are known to be major reservoirs of coronaviruses, and SARS COV-2 likely had its origins in some form from bats.

Why Don't Bats Get Symptoms of Viral Infection?

Although bats are carriers of these many viruses, including the coronaviruses, they do not show signs of infection, yet the same viruses are highly infectious to other mammals including humans when transmitted. Why is that? The first possibility is that there are special adaptations of the bat immune system, especially the innate immune system. Zhou et al (2015) has reported that in the Australian flying fox bat Pteropus alecto, the genome is highly compressed with respect to alpha interferon (IFN-a) genes (only 3 genes) vs. other mammals that typically have 7-18 IFN-a genes, and that unlike other mammals, IFN-a is constitutively expressed in the bat's unstimulated tissues. In humans, IFN-a (i.e., type I interferons, which includes IFN-a and IFN-beta) is stimulated to be expressed in response to detection of viruses by the innate immune system, but is not constitutively present. The always-on anti-viral activity of IFN-a in bats may be an adaptation to provide resistance to viral infections and circumvent the strategies that some viruses (including coronaviruses) have to inhibit or disable the innate immunity signaling pathways that ultimately would otherwise lead to IFN-a expression and downstream antiviral responses. There is evidence that Toll-Like Receptor 7 (TLR-7, innate immune system component, as discussed below) signaling is inhibited in humans by coronaviruses, potentially blocking the pathway to transcriptional activation (via interferon response elements, or IREs) of genes to produce type I interferons.

One conclusion from analysis of the difference between bat and human innate immune adaptations in bats vs. humans is that administration of alpha interferon or beta interferon, both of which are drugs in routine clinical use worldwide for other therapeutic indications, may be an effective treatment for COVID-19. Since in bats type I interferons are constitutively expressed, by analogy, this would suggest that administering alpha interferon or beta interferon to humans would best be done either prophylactically to high risk individuals or at the time of exposure to the virus or on initial infection. This would require the availability of a diagnostic screening test or method that would detect the SARS COV-2 virus rapidly after exposure or initial infection, or a decision and policy to administer IFN alpha and/or IFN beta to high risk individuals prophylactically.

One potential caution in implementing this strategy is that the ACE2 gene has been reported to contain an upstream gene sequence that binds STAT1, a transcription factor stimulated in response to alpha interferon, suggesting that ACE2 expression may be upregulated by interferon. Ziegler et al (2020) found evidence that alpha interferon stimulated increased expression of ACE2 in certain nasal secretory cells. In this case, interferon may increase the number of ACE2 targets available for viral attachment and infection. Whether this phenomenon leads to actual increases in ACE2 protein on key cell surfaces in COVID-19 infection or has an impact on the course of infection remains to be seen and even so may only come into play on more severe infections with more virus being present at the time of new synthesis of ACE2 protein. Furthermore, additional synthesis of ACE2 receptor during active infection may have a beneficial role in that internalization of ACE2 receptor by SARS COV-2 attachment and internalization reduces the amount of ACE2 present to perform its role in the renin-angiotensin system. ACE2 is critical for reversing the vasoconstrictive effect of angiotensin II in the bloodstream.

A second adaptation or ecological consequence for controlling virus infection symptoms, at least by fruit bats, may be related to their diets. Fruit bats eat bananas, mangoes, figs, avocados, and dates, in particular. Of these, all but dates (and they may too) are reported to contain significant levels of lectins, which are proteins that bind to specific sugar residues of glycoproteins. Lectins are generally resistant to stomach acids and digestive processes so they mostly remain in the digestive tract. Viruses, including coronaviruses, have surface proteins that are decorated with various sugar residues at the external most projections. Numerous plant lectins have been shown to specifically bind with patterns of sugar residues on virus coats. One possible consequence of the diet of fruit bats being high in lectins is that the lectins can bind up viruses in the gut and sequester them to be passed out in feces, limiting the amount of virus able to infect the host bat through entry in the small intestines. Some lectins can also pass through the tight junctions in the small intestines (leaky gut) and enter the bloodstream where they can also bind up viruses to limit infectivity toward the host cells.

For coronaviruses, the key external surface glycoprotein is the spike protein, which radiates out from the virus core in large numbers, giving coronaviruses their characteristic “corona” appearance. Although the putative source for the SARS and SARS COV-2 viruses is a horseshoe bat (i.e., not a fruit bat, but an insect eater), it has been demonstrated that a number of purified mannose-binding lectins, especially banana lectin, bind with high affinity to the SARS virus' spike protein, and in doing so are able to inhibit the virus in lab studies, as described further below. Insects contain lectins as well, possibly from eating lectin-containing plants. Lectins are considered an elementary form of innate immune defense used by plants and lower animals, and by mammals as well.

One novel conclusion from analysis of the diets of bats with respect to possible means to limit viral infectivity, may be to administer certain fruits or other foods rich in lectins, or other preparations containing such food-derived lectins, or purified or recombinant lectins by oral route as an effective treatment for COVID-19. In particular, for example, without limiting other options, consumption of bananas may be one preferred method for treatment of COVID-19.

Returning to the above discussion of a primary route of SARS COV-2 infections in humans potentially being the small intestines, consuming food containing molecules that can bind the virus in such a way that it cannot gain entry into the cells lining the small intestines could allow the immobilized virus to pass through the digestive tract, thus preventing or limiting the degree of infection. Preferably these binding molecules would have characteristics that allow then to survive passage through the stomach intact, be active in the lumen of the intestines, and be large enough (high molecular weight) not to pass through the gut into the bloodstream. Furthermore, preferably the lectins should be in sufficiently high concentration in the food material, and the active lectin itself should have a relatively high binding affinity for the SARS COV-2 virus. And further preferably, the binding should be to a portion of the SARS COV-2 virus, such as the spike protein, so that the virus's affinity to its infectious target (e.g., ACE2) or its ability to fuse with the endothclial cells of the small intestines is limited or prevented, either directly or sterically.

Two lectins in particular that bind to mannose sugars on surface glycoproteins of viruses and have been shown to possess antiviral activity are banana lectin (aka, BanLec) from bananas and plantains, and griffithsin, a lectin found in red seaweeds (Griffithsia sp). Another less relevantly documented but potential anti-viral lectin is cyanovirin-N from Nostoc species. Many additional lectins, especially lectins directed toward mannose residues on proteins, may be useful (which would need to be confirmed with respect to their binding to SARS COV-2), including but not limited to the following: leek lectin, mango lectin, pineapple lectin, lectins from other algae genera or species (such as Porphyra, Palmeria, Agardhiella, Gracilaria, etc.), soybean lectin, garlic lectin, snowdrop lectin, amaryllis lectin, lentil lectin, jacalin (lectin from jackfruit), etc. Some of these lectin sources may not be suitable for consumption without further safety studies although many of them are contained in widely consumed food sources.

Bananas, containing banana lectin, may represent an especially promising possibility for limiting infection of SARS COV-2 through the gut, both because banana is a widely consumed food and because its lectin has relevant attractive biochemical properties. Banana lectin is a dimeric protein, with each subunit having a molecular weight of 15 kD, and has a high affinity for binding to mannose and mannose containing glycoproteins. It is found at relatively high levels in the pulp of bananas (about 4 mg/100 g of pulp, with an average banana being about 100 g). Purified or recombinant banana lectin (or BanLec) has been shown to bind to the glycosylated surface protein GP120 of human immunodeficiency virus (HIV) (Swanson et al, 2010), and in doing so to strongly inhibit HIV viral entry into cells in culture. The effect is very potent, with an IC-50 value in the low nanomolar to picomolar range. Hopper et al. (2017) modeled BanLec binding mechanisms with HIV and found BanLec assembled into tetramers with multiple binding sites on GP120, leading to aggregation of virus protein and again blockage of viral entry. Coves-Datson et al. (2019) demonstrated that a recombinant variant of BanLec (H84T) inhibited the Ebola virus by similar mechanisms. Furthermore, the BanLec variant was administered to mice (intraperitoneally) that were challenged with an otherwise lethal dose of Ebola. Partial protection (50-80% survival) was achieved in the treated mice, including mice that were pretreated before challenge. Keyaerts et al., 2007, tested 33 plant lectins (but not BanLec) in a SARS virus infectivity test in vitro and found 10 lectins that were inhibitory, with leek (mannose-targeted) lectin having the most potency of high nanomolar EC-50. Similar to HIV and BanLec, the mechanism identified was binding to the coronavirus envelope (Spike) protein and preventing entry into the cell, as well as limiting exit of new virus particles from the host cell. These datapoints suggest banana lectin, and potentially other mannose binding lectins, could have an inhibitory effect on SARS COV-2.

Some potential routes of administration and doses of banana lectin could be to consume one medium sized banana each in the morning, noontime, and evening. Since it takes 4-6 hours for food to clear the small intestines, this dosing could potentially provide for nearly continuously presence of banana lectin in the small intestines during the daytime, the period of likely virus exposure. Because of the fairly high levels of banana lectin a banana, one banana may theoretically be an effective dose. For example, 4 mg of banana lectin (per banana) with a MW of the dimer of 30,000 would represent 1.33×10⁴ moles. Dividing by Avogadro's number yields about 10¹⁶ molecules equivalent. An average coronavirus particle has 74 spike proteins (Wiki), which at a 1:1 binding stoichiometry with banana lectin (it is potentially 2:1 or 3:1 lectin per spike protein) would suggest the potential for one banana to bind about 10¹⁴ virus particles. Even if these input numbers or calculations are incorrect, the relative magnitude suggests a significant binding capacity per banana. Clinical trial experimentation by one ordinarily skilled in the art would help define an optimal consumption schedule and amount for effective dosing.

Human Lectin Pathway in Innate Immunity

Humans also use lectins as one component of their defenses against infectious agents. The innate immune system is designed to recognize foreign pathogens and form a first line of defense by the body in holding that pathogen at bay until the adaptive immune system can form a more permanent defense. This process eventually results in the production of neutralizing antibodies by the body (adaptive immune response) which can specifically identify, bind to, and mediate clearance of the targeted invading pathogen, in this case the SARS COV-2 virus. However, the first production of antibodies through the adaptive immune response generally doesn't occur until about one to two weeks after initial infection by a new pathogen naïve to an individual. Unfortunately, severe cases of COVID-19 can progress more rapidly to adverse outcomes, morbidity or death, than the timeframe for antibody appearance. A critical goal for mitigating COVID-19 should therefore be to control viral levels and viral infectivity until neutralizing antibodies can form and clear the infection. This should be the role of an effectively functioning innate immune system.

The innate immune system depends first on a pattern recognition receptor system to identify molecular or chemical structures (Pathogen Associated Molecular Patterns, or PAMPs) that are foreign to the body; e.g., only found in pathogens. One main system for pattern recognition is a series of toll-like receptors (TLRs) expressed on or in certain immune system cells, especially dendritic cells and macrophages. In humans, there are 10 different TLRs known, each having a specific type of molecular structure that they recognize. Two of these TLRs are TLR7 and TLR8. Both of these target single stranded RNA. The SARS COV-2 virus is a single-stranded RNA (ss-RNA) virus. Therefore, recognition of SARS COV-2 by TLR7 in particular should initiate an innate immune response against the virus. There is evidence from studies on SARS coronavirus that this process involving TLR7 is indeed initiated but potentially partially disabled by the virus.

(See Drawing #1)

TLR7 is constitutively expressed in the small intestines and colon, and present in endosomes in immune cells especially dendritic cells and macrophages. Its expression can also be induced in human airway epithelial cells and primary cardiac cells on infection by viruses. Once TLR-7 binds the targeted ss-RNA virus, a series of messages are communicated and amplified in the cell, mediated by attachment of an intermediary protein called MyD88 to TLR7, then a number of signaling pathways are stimulated to produce an innate immune response to the virus. For TLR7 signaling these pathways can lead to production and release of type I interferons (IFN-alpha and IFN-beta), which have antiviral activity, in which case the pathways leading to stimulation of interferon-producing genes include an intermediary signaling protein called TRAF (TRAF3 or TRAF6). Alternative TLR/MyD88 innate immune responses can trigger a more complex signaling pathway, which also passes through TRAF6, leading to activation of the transcription factor Nf-Kb. In turn, Nf-Kb turns on production and release of pro-inflammatory cytokines including interleukin-6 (IL-6) and IL-12. TLR-8 signaling also uses TRAF3 as a pathway component leading to stimulation of production of type-1 interferons. These complex reactions constitute a major component of the innate immune response that can have a direct antiviral effect to dampen the degree of infection plus lead to presentation of viral antigens needed for the longer term adaptive immune response.

There is evidence (Li et al., 2016) that the papain-like protease (PLPro) produced by the SARS coronavirus as an initial step in replication after the virus has infected a cell can disable TRAF3 and TRAF6 by removing the ubiquitin chains on these signaling proteins at a specific site (Lys63). This results in a reduction in type 1 interferon production by the host organism. Furthermore, another innate immune system pathway that responds to single stranded RNA viruses, mediated by retinoic acid inducible gene 1 (RIG-1), also can be disabled by the SARS virus. The nucleocapsid protein (N) of SARS was shown (Hu et al., 2017) to bind to a motif of the protein TRIM25 which normally activates RIG-1 after RIG-1 recognizes its RNA PAMP. The TRIM25-RIG-1 reaction is also mediated by ubiquitin modification. The SARS N protein blocks this interaction, inhibiting the RIG-1 pathway which would otherwise lead to interferon production.

Based on this potential blockage of type 1 interferon production by coronaviruses, assuming the same mechanisms hold true with the SARS COV-2 virus, the administration of interferon alpha or interferon beta as a prophylactic or early intervention for COVID-19, as suggested above with respect to mimicking bats' always-on interferon adaptation, could be reinforced as a prophylactic or therapeutic strategy. There have been anecdotal reports of beta interferon having a positive effect in COVID-19 patients if administered early upon infection.

The Lectin Pathway and Complement System

Another set of pathways involved in the innate immune system is the complement system, including the traditional pathway, the alternative pathway, and the lectin pathway. These pathways have different mechanisms of initiation but all converge to a common intermediate in the pathways of complement at the protein complement C3, which is the point at which the downstream complement system gets activated. Of these pathways, one potentially most relevant to COVID-19 is the lectin pathway, in particular the component of that pathway based on initiation by Mannose-Binding Lectin (“MBL”, also known as mannan-binding lectin).

Like the TLRs, the role of mannose-binding lectin (MBL) in the innate immune system is to recognize specific molecular structures (PAMPs) of pathogens. In the case of MBL, it recognizes and binds to specific sugar residues of especially mannose, as well as N-acetylglucosamine (GlcNAc), on the glycoproteins of viruses, bacteria, etc. These sugar residues are generally not common on normal human proteins, but do appear over time on some damaged or diseased cells in the human body, which in this case are called DAMPs (Disease-Associated Molecular Patterns). MBL is produced in the liver and circulates in the bloodstream as a complex with two trypsin-like serine proteases called MBL-associated serine protease-1 (MASP-1) or MASP-2. MBL can also circulate in a complex with a truncated form of MASP-2 which lacks the serine protease activity, called MAP19, or MBL can circulate uncomplexed. When MBL binds to a mannose (and/or GlcNAc) residue on a pathogen, MASP-1 and MASP-2 are activated (MASP-1 is thought to activate MASP-2, although both MASP-1 and MASP-2 can autoactivate), and that triggers a number of downstream events. Activated MASP-2 can generate the complement C3 activating protease, C3 convertase, by cleaving C2 and C4 to form C4b2a, thus activating the rest of the complement cascade. The MBL-pathogen complex (connected at the mannose binding site to pathogen's mannose-containing glycoprotein) can be marked by deposition of complement C4 which acts as a direct opsonin to be recognized by antibodies of the adaptive immune system and eliminated. Following C3 activation by MASP-2, the anaphylatoxins C3a and C5a can be generated, both of which are pro-inflammatory mediators. And the terminal component, Complement 5a-9, can form the Membrane Attack Complex (MAC) that lyses damaged cells or pathogens that had been marked in the opsonization process. (review by Noris and Remuzzi, 2013).

(See Drawing #2)

MBL has been shown to bind to the SARS virus, through mannose residues on the Spike protein. The binding of MBL to SARS virus appears to sterically hinder the ability of the SARS virus to infect host cells, possibly changing the Spike protein conformation or shielding the S1/S2 or S2 cleavage sites from proteases that would otherwise facilitate fusion. Molecular modeling of SARS COV-2 suggests that its Spike protein contains most of the same glycosylation sites as the SARS spike protein plus as many as four new glycosylation sites. Therefore, it is likely that MBL binds to SARS COV-2 as well, perhaps to a different degree or different or additional site(s). The lectin pathway, specifically MBL, may be a key early antiviral response mechanism to fight off COVID-19, potentially in individuals who have been infected but are asymptomatic or have mild disease.

MBL is encoded by the mbl2 gene. There are several known mutations or single nucleotide polymorphisms in the promoter and coding regions of the mbl2 gene that result in no or reduced levels of MBL in circulation (Garcia-Laordin et al., 2008). Individuals with these insufficient levels of MBL are significantly more prone to severe infections, including pneumonia and other lung infections, HIV, and respiratory infections in children, and to impaired lung function in cystic fibrosis. Ip et al. (2005) analyzed more than 500 patients who had SARS vs. over 1000 controls and found that individuals with low-MBL mbl2 polymorphisms were over-represented in the SARS group, and that SARS patients had lower average levels of MBL protein in blood vs. the control group. The prevalence of MBL deficiency due to mbl2 polymorphisms is estimated to be 5-10% in world populations, with a higher incidence in individuals from sub-Sahara Africa and their descendants. Assuming MBL is a key player in the innate immune system antiviral defense against COVID-19, like in SARS, then individuals with these mbl2 polymorphisms may account for some of the cases of higher susceptibility, severe morbidity, or death among COVID-19 patients, and/or may account for some of the incidence of symptomatic or severe cases in otherwise healthy younger patients. Genetic testing of individuals for these mbl2 polymorphisms should be considered for prescreening for the potential of a more severe respiratory infection from SARS COV-2. In addition, MBL levels in blood should be measured in COVID-19 patients as soon as possible after positive diagnosis to assess the potential for more severe infections in individuals with low or no serum MBL protein levels.

On the other hand, there are individuals with above normal levels of MBL circulating in blood. Such cases are often associated with the “DAMP” side of the lectin system. As part of the complement system attack against pathogen infected cells, MBL-marked cells are targeted for destruction by the immune system. If normal human cells or normal glycoproteins are modified through a disease process to aberrantly have more sugar residues such as mannose or N-acetylglucosamine, they can be marked for destruction as well. One major disease that appears to have an association with MBL is diabetes. MBL levels are elevated in Type 1 diabetic patients, especially those with diabetic nephrology and diabetic retinopathy. In addition, levels of MASP-1 and MASP2 are elevated in diabetics and correlate with diabetic control (Jenny et al., 2014). In diabetes, glucose is not fully utilized by cells in the body, either as a result of insufficient production of insulin by the pancreas or reduced ability by cells to take up glucose. As a result, excess sugar builds up in the bloodstream and is deposited on proteins as additional glycosylation. One protein on which excess sugar is deposited is hemoglobin, which carries oxygen through the bloodstream in red blood cells and removes CO₂. Diabetic control is often measured by the amount of glycosylation of a hemoglobin type called Hemoglobin A1c, for which measurement of incorporated sugars rises over time in uncontrolled diabetics. There are other hemoglobin types besides A1c. Some hemoglobin has been shown to contain excess mannose residues, and/or are modified with N-Acetylglucosamine, which can be recognized by MBL. With elevated MBL levels in diabetics, the hemoglobin with aberrant mannose levels may be more rapidly tagged for destruction by the complement system. Normally, red blood cells containing the hemoglobin last for about 3-4 months before they are naturally degraded by macrophages, which occurs in the spleen or liver. One of the byproducts of hemoglobin degradation is bilirubin. Red blood cells with hemoglobin are gradually replenished from hematopoietic stem cells by stimulation with erythropoietin.

MBL response to a PAMP is an acute phase reaction following infection. As MBL is bound to the pathogen, levels of free MBL in the bloodstream are initially reduced and expression of additional MBL complex is upregulated. As virus titers increase due to viral replication, presumably more MBL is produced as well. If the additional MBL is targeting both SARS COV-2 and PAMPs such as heavily glycosylated hemoglobin and/or red blood cells, then both positive and negative effects of the MBL directed innate immune response may be expected.

First, on the positive side, with the TLR-7 mediated innate immune response potentially disabled by SARS COV-2 to prevent or limit type 1 interferon production, MBL may be the key to early control of systemic COVID-19 infection by the innate immune system. MBL bound to the SARS virus prevented infection of cell lines in in vitro studies, apparently by blocking the cell fusion step, possibly by hindering the binding of the activating serine protease (e.g., TMPRSS2). MBL's opsonization response may also be the stimulus for the adaptive immune system to kick in with an eventual neutralizing antibody response. Complement C4, the complement of the lectin pathway deposited on pathogens to stimulate their destruction, has been identified deposited on SARS virus in studies (Ip et al., 2005). Therefore, the early response by MBL should be allowed or facilitated, not blocked.

On the negative side, in cases of more severe infections or in patients in which there is a predisposing condition involving DAMPs recognized by MBL, the outcome may turn to an adverse morbidity. For example, in diabetics, if aberrant glycosylated hemoglobin is cleared more rapidly, and replenishment from hematopoietic stem cells can't keep up, hemoglobin levels in patients may drop. This would lead to a reduction in both oxygen supply to tissues and removal of waste CO2. Many severe COVID-19 patients have been reported to have low oxygen saturation in the blood and yet apparently intact functioning lungs not characteristic of ARDS (acute respiratory distress syndrome). A reduction in hemoglobin levels could explain these symptoms. Further supporting this possibility, hemoglobin levels as low as 50% of normal have been reported in severe patients, and elevated bilirubin levels are being reported in individuals. In a study (Richardson et al., 2020) of 5700 hospitalized COVID-19 patients in New York, average ferritin levels, a marker for anemia or low red blood cells or excess destruction of red blood cells, were elevated. LDH was also elevated, a sign of low oxygen levels as pyruvate is converted to lactose. A common symptom of COVID-19 is patients gasping for air as if they did not have enough oxygen, and feeling tired or lethargic, which also could result from low oxygenation. However, attempts to re-oxygenate patients through the lungs via mechanical ventilation has not been very successful. (The study of 5700 hospitalized COVID-19 patients in New York reported a mortality rate of 88% in the subset of those patients who were put on ventilators.) These factors suggest the lack of oxygenation in patients was mainly due to an internal systemic dysregulation rather than a respiratory infection-induced blockage of the lungs. Furthermore, it has been widely reported that diabetes, as well as obesity, often considered a prediabetes condition, are major risk factors for severe outcome in COVID-19, again making a connection between MBL/MASP-2 activation and degradation of red blood cells and 02 carrying hemoglobin.

As a further tie to adverse events in COVID-19 infection, the lectin pathway, which is a primitive immune response mechanism against pathogens found in many lower organisms, has activity in promoting blood coagulation as well as the innate immune response. There is speculation that lower organisms used the lectin pathway to coat pathogens with fibrin, a component of blood clots, as a means of immobilizing the pathogen and limiting the spread of infection. It has been shown that the serine protease MASP-2 can directly cleave prothrombin to generate thrombin, which in turn converts fibrinogen to fibrin, contributing to coagulation of blood. In addition, MASP-1 can directly cleave fibrinogen to form fibrin, and can activate Factor VIII, a protein in the coagulation cascade. These mechanisms lead to fibrous fibrin deposition on blood clots and on damaged tissues or cells, which in turn could lead to such adverse events as deep vein thrombosis, myocardial infarction, disseminated intravascular coagulation, and conditions similar to manifestations of Kawasaki disease. These are all conditions that have been reported in some more severe COViD-19 patients. The potential continuous stimulation of clot formation and fibrin deposition may lead to countervailing attempts by the body to dissolve the clots, leading to breakdown of clot components as well. Levels of D-dimer, a breakdown product of fibrin clots, are elevated on average in hospitalized COVID-19 patients, and correlate with higher probability of death. Other coagulation-related lab findings in COVID-19 patients include thrombocytopenia and prolonged prothrombin-time.

The following are some reports of links between coagulation system disorders or conditions and the lectin pathway/MBL/MASP-1/MASP-2:

“Simultaneous Activation of Complement and Coagulation by MBL-Associated Serine Protease 2” [MASP-2], Krarup, A. et al., PLosONE 7:e623 (2007)
“Activation of mannan-binding lectin-associated serine proteases leads to generation of a fibrin clot”, Gulla, K. et al., Brit. Soc. Immunology 129:482-495 (2009)
“Plasma levels of mannose-binding lectin and future risk of venous thromboembolism”, Liang et al., J. Thromb. Haemost., 10:1661-1669 (2019)

### “MASP-1 Induced Clotting—The First Model of Prothrombin Activation by MASP-1”, Jenny et al., PLOS One, pp 1-13, (Dec. 8, 2015)

The following paper discusses the role of MASP-1 in dissolution of clots as well:

“MASP-1 of the complement system alters fibrinolytic behavior of blood clots”, Jenny et al., Mol. Immunol.114:1-9 (2019)

(See Drawing #3)

The constant stimulation of the MBL/lectin pathway upon increasing titers of SARS COV-2 during infection may result in an over-activation of the coagulation system by the presence of increasing levels of MBL-associated MASP-1 and MASP-2. The MASP-1 and MASP-2 induced fibrin deposits can then continue to grow into larger clots raising the potential for adverse cardiovascular events. Furthermore, since ACE2 is expressed in the endothelial layers of blood vessels, MBL/MASP1/MASP2 attached to the virus at sites of infection in the blood vessels could lead to microclots on the vessels such as seen in disseminated intravascular coagulation (DIC), as well as vasculitis. This condition manifests itself as red splotches or rashes visible on the skin. It can also be seen in discoloration of the extremities such as toes and fingers, along with inflammation. Similar symptoms can be seen in Kawasaki's Disease and/or Heinoch-Schoenlein Purpura (HSP), both of which have been linked with infectious diseases.

In April and early May 2020, there were increasing reports of COVID-19 positive patients exhibiting symptoms similar to those described above, especially in children. The MBL-based coagulation activation could be the cause of these symptoms. A further complicating factor for this syndrome may be these patients having defective components of the antithrombotic control mechanisms that would normally limit the extent of fibrin deposition and clot formation. One such defect may be Factor V Leiden, an inherited mutation in the Factor V gene that reduces normal Factor V's ability to feed back and inhibit the production of thrombin from prothrombin, thereby stopping further clot formation. Specifically, Protein C, a natural coagulation inhibitor, is unable to bind to Factor V due to the mutation, so that Factor V fails to inhibit the pro-thrombin to thrombin step. Factor V Leiden polymorphism is found in about 5% of Caucasians in North America, and potentially higher incidence in Europeans. It is rare in other ethnic groups or races. Some of the cases of the “Kawasaki-like” syndrome being reported in otherwise healthy children with COVID-19 may be a result of Factor V Leiden mutations. A genetic test is available for Factor V Leiden mutation and may be a valuable screening tool for newly diagnosed COVID-19 patients.

Many of the reports of the cardiovascular complications from COVID-19 are in patients that have been infected for a period of time, such as a week or more, suggesting that the steps leading to the cardiovascular complications are fairly gradual. This means that there is time after infection for therapeutic intervention. It also means that if the initial symptoms of COVID-19 (fever, for example) resolve quickly, there still may be a risk of progressive deterioration in non-direct viral infection complications such as hypercoagulability, and intervention still may be warranted.

Direct treatment for these coagulation complications after they occur could be by administering anti-thrombin plus heparin, both of which are approved medications. Anti-thrombin binds to thrombin and to Factor Xa, thus inhibiting fibrin deposition. Heparin enhances the binding affinity of anti-thrombin to its targets. Other forms of anticoagulant therapy may be warranted, but given the varied dynamics of clot formation and fibrin dissolution, optimal agents and doses would need to be determined.

An inhibitor of MASP 1 and/or MASP-2 could be a valuable therapeutic option for limiting the coagulation cascade. An inhibitor of MASP-2 (antibody to MASP-2) is in Phase III clinical trials for treating hematopoietic stem cell transplant-related thrombotic microangiopathy (TMA). TMA is a disease or condition in which blood clots form in small blood vessels or capillaries, leading to damaged epithelium, stopped or reduced blood flow, and deformation and bursting of red blood cells. The same antibody drug is also in Phase III trials for treating Hemolytic Uremic Syndrome (HUS), which usually occurs in children in response to an infection or bacterial toxin. HUS is also characterized by endothelial damage especially in the kidney, clotting, and burst red blood cells. These conditions seem to correspond to many of the adverse cardiovascular and coagulation symptoms seen in severe COVID-19 patients.

An inhibitor of Complement C3 is in Phase III clinical trials for treating paroxysmal nocturnal hemoglobinuria, which, in addition to its primary symptom of destruction of red blood cells due to excessive complement activity, is characterized by a high incidence of clot formation. Therefore, this agent could also have a dual role of limiting some or many of the coagulation-related adverse effects of COVID-19, as well as potentially limiting the adverse effects of low oxygen supply and reduced hemoglobin/red blood cell levels from excess or aberrant complement-mediated damage in subsets of patients with COVID-19, as discussed above. However, since C3 falls lower in the complement cascade than MASP-1/MASP-2, and because C3 inhibition would be less likely to have a direct effect on the coagulation activation component, an inhibitor of MASP-1/MASP-2 would likely be a superior choice in treating COVID-19.

One well known condition following a heart attack or other cardiovascular event is reperfusion injury. As oxygen and blood flow is restored, serious complications can continue as the damaged cells are removed and destroyed by the complement system and other immune cells. There have been some reports of COVID-19 patients having adverse cardiovascular events after apparent recovery from the viral infection, which could potentially be related to this condition. Therefore, treatment with MASP-2 or C3 inhibitors or otherwise blocking the MBL pathway following infection and onset of symptoms could still be beneficial, and perhaps even after clearance of the SAR COV-2 infection by a patient, as suggested by the following study.

In a study with transgenic mice expressing human MBL (hu mbl2 knock-in) (Jordan et al, 2001, see also Pavlov et al., 2015), an antibody that blocked MBL administered to mice after an induced myocardial infarction and initial reperfusion showed the following: preserved myocardial function, reduced infarct size, prevented fibrin deposition within the myocardium, and prevented occlusive arterial thrombogenesis. These positive effects were all seen with MBL antibody treatment after the cardiovascular event, suggesting that an MBL/MASP-2 inhibition after the onset of SARS COV-2 infection might still be beneficial to resolve symptoms.

As further possible suggestion for the involvement of the lectin pathway and/or specific glycoprotein sugar residues in COVID-19 susceptibility and morbidity, in a case study in China (Zhou et al., 2020), significantly higher risks of infection and death were found to be associated with blood type A patients, and significantly lower respective rates were observed in blood type O patients. People with blood type A have the A antigen expressed on the surface of their red blood cells. “A” antigen is formed by first the enzyme fucosyltransferase adding fucose sugar residues to surface proteins on cells to form the H antigen, then the enzyme glycosyltransferase adds N-acetylglucosamine moieties on the deposited fucose (the glycoprotein is now called the “A” antigen). MBL binds N-acetylglucosamine, in addition to mannose. People with “O” blood type do not have the A antigen on their blood cells but have circulating anti-A antibodies, which are not present in Type A blood patients. In a study with SARS, Patrice et al. showed that anti-A antibodies specifically inhibited binding of the spike protein of SARS to ACE2 receptor-expressing cell lines, suggesting a possible explanation for observed lower infection rates in individuals with type O blood, and again suggesting a possible positive role of MBL itself with respect to SARS COV-2.

If this disease progression is correct, then potential therapeutic options could include the following:
1. Using a diagnostic test that is accurate and detects early infection and/or viral titers in patients, taking no intervention with respect to this mechanism for a period after infection (or at below a certain threshold of viral titer) that would allow the lectin pathway/MBL to develop an initial innate immune response to SARS COV-2. Based on the general guidance that symptoms do not develop until about 5 days after initial infection, no intervention may extend for a period of several days into the infection. Thereafter, the treatment options might be:
   An inhibitor of the serine proteases MASP-1 or MASP-2 that would stop the complement cascade at the beginning of the process. A natural inhibitor of MASP-1 is sunflower trypsin inhibitor (SFTI), found in the seeds of sunflowers, and other Bowman-Birk inhibitors. An antibody against MASP-2 (narsoplimab; OMS-721) is in Phase 3 trials for treatment of hematopoietic stem cell transplant-related thrombotic microangiopathy, a disease in which endothelial dysfunction leads to microangiopathic hemolytic anemia, platelet activation, and formation of platelet-rich thrombi. Since MASP-2 is the only enzyme of these two shown to have the ability to autoactivate the complement cascade, MASP-2 blocker would be the preferred target of these two.
   An inhibitor of complement C3 that would stop the complement cascade at the junction of the three different complement pathways. A C3 inhibitor (Pegcetaclopan) is in Phase 3 trials for treating proximal nocturnal hemoglobinuria (PNH), a rare genetic disease in which hemoglobin levels are depressed.
   An inhibitor of complement C5, which is one of the final components in the complement cascade and is associated with a pro-inflammatory response. A C5 inhibitor (eculizumab, or Soliris) is a marketed drug for treating PNH. This approach may be too late in the complement cascade to counteract some of the potential mechanisms discussed above, but could reduce symptoms of excessive inflammatory response.
   An inhibitor of the receptor for complement C5a (Avacopan; CCX-168) is in Phase III trials for treating ANCA associated vasculitis, which is an inflammation of the small blood vessels in the body caused by anti-neutrophilic cytoplasmic autoantibodies. Blocking the C5a receptor could reduce some of the pro-inflammatory consequences of complement system activation but as a late cycle mediator may not be as relevant as an earlier stage inhibitor of the MBL pathway.
2. Administering potential blockers of the interaction between MBL and its mannose or N-acetylglucosamine targets on glycoproteins such that these mimics of the MBL target are occupied without activating MASP-1 or MASP-2. These might include D-mannose and other small sugar derivatives that bind to MBL competitively with its glycoprotein targets.
3. Administering mimics of the mannose-binding function of MBL that cannot complex with MASP-1 or MASP-2 to activate the complement system. Specifically this might include mannose lectins that were mentioned above as potential treatments or prophylactics by oral consumption to prevent SARS COV-2 from entering through the small intestines, such as banana lectin, or griffithsin, or lower molecular weight fragments of such lectins that would retain the mannose binding capability, or other similarly functioning molecules. These lectins would be administered systemically rather than (or in addition to) the proposed oral consumption route. For example, griffithsin, a 12.7 kD protein isolated from a red algae with strong mannose-binding properties, has demonstrated potent in vitro and in vivo antiviral activity against SARS virus (O'Keefe et al., 2010). The protein is being produced in genetically engineered tobacco plants for clinical testing as a microbiocide against HIV transmission. Derivatives of griffithsin with lower molecular weight and/or altered pharmacologic or pharmacodynamics properties have been produced including grifonin-1 (Micewicz et al., 2010). Similarly, an altered form of banana lectin, called H84T, has been developed that eliminates a mitogenic activity of banana lectin (Swanson et al., 2015) while preserving its antiviral properties, specifically against Ebola (Coves-Datson, 2019).
   Another option might be modified MBL that cannot complex with MASP-1/MASP-2. A recombinant MBL was previously produced but apparently discontinued in clinical studies. In a preclinical study, mice administered high dose rMBL (7× that normally in human serum) survived otherwise fatal challenge with Ebola virus and became immune to virus re-challenge (Michelow et al., 2011). Whether this form of MBL complexed with MASP-1/MASP-2 after it was administered, or would be in humans, is uncertain.
4. For non-drug medical intervention, steps to replenish functional hemoglobin and red blood cells should be undertaken as soon as soon as possible and be sustained. This could include administering erythropoietin (marketed drug) and/or hematopoietic stem cells. Erythropoietin may have a lag of several days before a significant increase in mature red blood cells is seen. Blood transfusions could also be used as an immediate step, especially in severe cases of reduced hemoglobin levels.

One further possibility that should be explored experimentally is whether MASP-1 or MASP-2, both trypsin-like serine proteases, can cleave the SARS COV-2 spike protein at S1/S2 and/or the S2 site, as has been shown for TMPRSS2, another trypsin-like serine protease. In such case, MBL could bind SARS COV-2 in a position to be activated by MASP-1 or MASP-2. This could result in activated SAR COV-2 circulating in the bloodstream, or SARS COV-2 attaching to the ACE2 receptor in a pre-activated site for entry into a host cell without the need for TMPRSS2 co-expression. If this were the case, it is likely that a MASP-1 and/or MASP-2 inhibitor would be a direct treatment for SARS COV-2 infection, not just COVID-19 secondary effects.

In this regard, again if this hypothetical activation phenomena as mentioned above is demonstrated, an inhibitor of MASP-2 (narsoplimab) in Phase III clinical trials for other indications could be a potential treatment option to block the serine protease mediated activation of SARS COV-2. With respect to blocking MASP-1, and potentially more generally blocking other serine proteases that may be able to activate SARS COV-2, administration of plant-based serine protease inhibitors (serpins) or derivatives thereof may potentially be effective antiviral treatments for SARS COV-2. Slightly modified forms of sunflower trypsin inhibitor-I (SFTI) were shown (Heja et al., 2012) to potently inhibit the activity of both MASP-1 and MASP-2 (with higher inhibitory activity against MASP-1). SFTI-1 is a 14-mer cyclic peptide found in relatively high levels in sunflower seeds. There have been numerous efforts to use SFTI-I as a scaffold to synthesize novel serine protease inhibitors. If MASP-1/MASP-2 does activate SARS-COV-2 for cell fusion similar to the reported role of the serine protease TMPRSS2, and SFTI-1 or other plant serpins can inhibit these serine proteases, then they may be able block SARS COV-2 infection. Even if MASP-1/MASP-2 are not involved, SFTI-1/plant serpins may be useful for blocking TMPRSS2 or furin or other serine protease activators of SARS COV-2. SFTI-1 could potentially be delivered orally via consumption of sunflower seeds in an amount and on a time schedule of dosing that one skilled in the art could determine. Since sunflower seeds are eaten by humans as a common food source, adverse events are unlikely. The sunflower seeds/SFTI-1 or other serpins could limit the infectivity of SARS COV-2 in the gut and/or may be absorbed into the bloodstream via the gut to act systemically. Consumption of sunflower seeds could be combined with consumption of mannose lectin sources such as bananas, as previously discussed. Banana lectin could bind to the SARS COV-2 spike protein and limit infectivity through a different mechanism of preventing binding or fusion with a host cell by steric hindrance or other mechanism. Alternatively, SFTI-l/derivatives/alternative plant serpins and/or banana lectins/derivatives/alternative mannose lectin sources could be formulated as drugs to be administered systemically via injection, infusion, or intravenously.

A recent related but slightly different mechanism for MBL and SARS COV-2 interaction that has been proposed (Gao et al, unreviewed preprint 2020) is that MBL can bind to the nucleocapsid glycosylated protein (N) of SARS COV-2, either separately from, or in addition to, its binding to the spike protein. The binding of N and MBL activates MASP-2, and then SARS COV-2 infects the host cell and replicates. When the new viral particles are released, N proteins are also released into the bloodstream, which are bound by more MBL, activating MASP-2, and triggering further complement activation. This creates a positive feedback loop from infection that multiplies the degree of complement activation and resultant pro-inflammatory response, leading to worsening secondary symptoms in COVID-19 patients. If this mechanism is correct, treatment with a blocker of MASP-2 would be a preferred therapeutic approach.

(See Drawing #4)

Implications of MBL-Associated Effects for Vaccine Development

Based on the assumption that molecular structures (PAMPS) on the SARS COV-2 virus (Spike and/or Nucleocapsid proteins, including their glycosylation sites) may be inducing lectin pathway-mediated immunological responses plus adverse coagulation system effects, care must be taken to ensure that any vaccine developed against SARS COV-2 not stimulate any adverse coagulation system effects after administration. Coagulation markers should be monitored in those individuals participating in clinical trials of vaccine candidates, in tandem with the usual immunological goals of looking for neutralizing antibodies and/or T-cell responses. Furthermore, the impact on more vulnerable population subsets, potentially including those individuals who have the Factor V Leiden mutation or other relevant polymorphisms or risk factors noted herein, should be evaluated during vaccine safety testing.

The ACE2 Receptor is a Critical Component of the Renin-Angiotensin System that Regulates Blood Pressure and Other Key Functions in the Body

Another set of cardiovascular adverse events, besides MBL-mediated coagulation and inflammatory effects, is likely due to the SARS COV-2 binding to its ACE2 receptor target. ACE2 is an important component of the renin-angiotensin system in the body, which is a key regulator of blood pressure and many other critical metabolic functions in humans. In this system, Angiotensin Converting Enzyme-2 (ACE2) converts angiotensin II (Ang-II) into angiotensin 1-7 (Ang 1-7) through proteolytic activity. The Ang II substrate for ACE2 is produced by the enzyme Angiotensin Converting Enzyme-1 (ACE1) from ACE1's substrate angiotensin I (Ang-I). Ang II has vasoconstrictive effects on blood vessels. Ang 1-7 has vasodilatory effects on blood vessels. So the relative levels of functional activity of ACE1 and ACE2, and corresponding amounts of circulating or locally produced Ang II and Ang 1-7, play a significant role in regulation of blood pressure, and in the degree to which blood vessels are constricted (narrowed, higher blood pressure) or dilated (wider, lower blood pressure). This relationship is targeted in the class of drugs called ACE inhibitors, which block ACE1 (not ACE2) to lower blood pressure by reducing the amount of Ang II in the bloodstream through decreasing the enzymatic activity of ACE1. Another drug strategy is blocking the receptor for Ang II, called Angiotensin I Receptor (AT1R), which is similarly an avenue for modulating the effects of excess Ang II in the bloodstream. Excess or hyperactivity of Ang II is associated with diseases including cardiac hypertrophy, heart failure, stroke, coronary artery disease, and end-stage renal disease.

Ang 1-7 generated by ACE2 activity has a relatively short half-life in the bloodstream (minutes), so its immediate effects are short-lived. However, Ang 1-7 binds to the Mas receptor (a G-Protein Coupled Receptor) thereby mediating the key vasodilatory and anti-inflammatory effects of Ang 1-7. The Mas receptor is located on cells of cardiac tissue, kidneys, and especially the brain, and is associated with the protective responses to counteract Ang II.

When SARS COV-2 binds to the ACE2 receptor and becomes activated for fusion with the host cell by the action of TMPRSS2 or another serine protease, the complex of ACE2 and SARS COV-2 (based on SARS findings) becomes internalized into that cell. This removes cell surface ACE2 that would otherwise be able to convert Ang II to Ang 1-7. The result is presumably vasoconstriction, especially in the region where ACE2 would otherwise exert local counterbalance to that effect if it were still present on the cell surface. As noted, ACE2 is heavily expressed in the lower lobes of the lungs, in the type 1 and type 2 alveolar cells. These are the primary cells through which O₂ and CO₂ are exchanged by flow (i.e., of oxygen) from the lungs to red blood cells containing hemoglobin passing through small blood vessels (capillaries) that bridge the venous and arterial sides of the cardiovascular system in the lung tissue. The capillaries are small so that red blood cells pass through almost single file, helping to maximize gas exchange with air in the lungs. If those capillaries become more highly constricted due to higher levels of Ang 11 and reduced levels of ACE2, then the red blood cells would have a more difficult time passing through the capillaries. Furthermore, if an individual had arthrosclerosis from deposits lining the vessels as a pre-existing condition, passage of red blood cells would presumably be even more difficult. And individuals with sickle cell anemia, in which red blood cells are deformed or stiffened so that even under normal conditions they are prone to having difficulty getting through capillaries, could be especially at risk for blood flow complications. Besides red blood cells, white blood cells such as platelets flow through the blood vessels including the capillaries. Platelets can stick to damaged or inflamed walls of a blood vessel and initiate clot formation.

(See Drawing #5)

The end result of SARS COV-2 infection of the alveolar cells in the lower lungs could well be such extensive vasoconstriction and inflammation that blood flow through the capillaries gets blocked and/or that clots form or dislodged venous clots formed elsewhere get caught in the narrowed arteries, thus having the same effect of blocking blood flow. This phenomenon could potentially account for areas of “ground glass opacity” (GGO) seen in lung scans of severe cases of COVID-19. One cause of GGO is pulmonary embolism. The potential pro-thrombotic effects from MBL-associated activation and deposition of fibrin in the bloodstream could further exacerbate this predicted vasoconstriction-induced capillary blockage. Reduced or curtailed blood flow through sections of lung capillaries could account for the hypoxia reported in many severe cases of COVID-19, as well as decreased red blood cell counts and lymphopenia as white blood cells get blocked from circulation too. Pulmonary embolisms that have been reported in COVID-19 patients would fit this mechanism. It could also help explain why the following are high risk, comorbidity factors for developing severe COVID-19: cardiovascular disease, hypertension, chronic stroke, and chronic lung disease. If eventually the capillary blockage exceeds a certain threshold of lung section blockage, systemic hypoxia can become so severe that organ failure and death result.

This phenomenon could also help explain some of the observed demographic aspects of severe and fatal COVID-19. Individuals with sickle cell disease have a mutation in one (sickle-cell trait) or both (sickle cell disease) of the beta-globin genes that make up hemoglobin, resulting in stiff or deformed (sickled) red blood cells that have more difficulty passing through normal capillaries and therefore making these individuals more vulnerable to vaso-occlusive effects like SARS COV-2 may cause. Sickle cell mutation is most predominant in sub-Saharan Africa and Eastern Asia. In the U.S., it is estimated that 1 in every 365 African American children have sickle cell anemia, while the frequency in Hispanics is 1 in 16,500 and in Caucasians far lower than that. An estimated 100,000 in the U.S. have the severe form (both globin units mutated), while another 2 million are carriers (single mutated unit). About 8% of African Americans are carriers. The likelihood of individuals with sickle cell having greater possibility for severe COVID-19 if infected, coupled with the relative predominance of this mutation in African Americans, could account in part for the higher relative proportion of severe cases of COVID-19 seen in African Americans vs. Caucasians. Given these factors, individuals diagnosed with COVID-19 should be tested for the presence of the sickle cell mutation and positive cases managed appropriately.

Another organ with a high density of capillaries is the kidney. ACE2 is also highly expressed in the kidney. ACE2 activity has been shown to be altered in diabetic kidney disease, hypertensive renal disease and in different models of kidney injury (Soler et al., 2013). In the kidney, capillaries mainly serve the purpose of transferring waste products out of the blood to be excreted. The same conditions as described above for the capillaries in the lungs could occur with respect to SARS COV-2 infection into kidney cells through binding to and internalization by the ACE2 receptor, followed by vasoconstriction. Markers for kidney dysfunction such as proteinuria and serum creatinine were elevated and glomerular filtration rates were depressed in about 13% of hospitalized COVID-19 patients in a study from China (Cheng et al., 2020). Chronic kidney disease or end-stage renal failure is a major risk factor for severe COVID-19. Diabetes, another major comorbidity factor for severe COVID-19, is considered a precursor of end stage renal disease. Damage to and failure of kidneys, including a not insignificant need for kidney transplants in the midst or aftermath of COVID-19 infection, have been reported with COVID-19. Furthermore, many individuals with kidney disease require dialysis on a regular (about every two days) basis. Without dialysis, kidney damage can be exasperated, yet access to dialysis machines in a COVID-19 ICU ward can be problematic, especially if the patient is put on a ventilator.

Can Reported Side Effects of ACE Inhibitors Explain Dry Cough Symptoms of COVID-19?

One of the reported side effects of taking ACE1 Inhibitors for treating hypertension in non-COVID-19 patients is a “dry cough”. A persistent, non-productive dry cough is considered a severe adverse event and the most common cause of withdrawal of ACE inhibitor use, occurring in 10%-20% of patients overall (Nishio et al., 2011). The incidence of dry cough in East Asians (e.g., Chinese) is substantially higher than in Caucasians, perhaps as much as two-to-three fold higher. A meta-analysis by Nishio et al. (2011) of polymorphisms in the alleles for bradykinin B2 receptor (BK₂R) and for ACE 1/D (D allele of ACE1) vs. the observation of dry cough side effect of ACE inhibitor use indicated a highly significant association between BK₂R polymorphism and dry cough in East Asians. The authors suggested that dry cough in ACE inhibitor patients may be due to mediators associated with the Renin-Angiotensin system but not directly to ACE1 blockage. It might be possible that the dry cough reported initially in Chinese COVID-19 patients was at least in part, and maybe significantly, associated with SARS COV-2 interaction with the ACE2 receptor, and less an indicator of a primary respiratory infection-induced symptom.

Potential Therapeutic Avenues for Treating Reduced-ACE2-Mediated Effects of COVID-19
1. Management of Cardiovascular Parameters. Given the adverse impact on the cardiovascular system from SARS COV-2 infection, a key support strategy for COVID-19 patients should be to apply or continue to apply standard measures to control blood pressure, clotting risk, vessel inflammation, etc. One area of concern has been whether COVID-19 patients taking ACE inhibitors should continue taking these drugs while infected with SARS COV-2. Administration of the ACE inhibitor lisinopril in rat studies led to a nearly 2 fold increase in circulating Ang 1-7 and a decrease in serum Ang II (Ferrario et al., 2005) both of which should be desirable outcomes to decrease vasoconstriction in COVID-19 patients. Tn these studies, cardiac mRNA for ACE2 was increased but ACE2 activity levels were not, at least in the time frame of the experiments. An increase in expressed ACE2 protein might not be desirable with respect to SARS COV-2 infection in that it might provide more sites for the virus to enter and infect cells. However, it would seem that a decrease in ACE1 activity should lead to no significant increase, and perhaps a decrease, in ACE2 protein levels or activity since less ACE1 means less substrate (Ang II) available for ACE2 to act upon. Therefore, it is likely that ACE inhibitor therapy should be continued and potentially initiated in COVID-19 patients. Recent studies in COVID-19 patients showed no additional adverse outcome for patients taking ACE inhibitors and in fact a better survival rate in one study among patients on anti-hypertensive drugs at the time of hospital admission.
2. Exposing the patient's blood to functional ACE2 protein that lacks the domains allowing the protein to embed at the cell surface and allow virus entry. Adding functional ACE2 would presumably result in more Ang II being converted to Ang 1-7 and consequently greater vasodilation. A secondary effect of this strategy might be binding up free virus in the bloodstream that then cannot infect a cell because the ACE2 is circulating free rather than being on a cell surface. Making and testing such a recombinant form of ACE2 would presumably take a relatively long period of time, and have unknown risks with respect to having the protein administered to a patient directly. An alternate means of carrying out this same strategy might be to expose a patient's blood extracorporeally to immobilized ACE2, using a combinatorial apparatus similar to a modified dialysis system containing a module with bound ACE2 past which the patient's blood flows, as further described below.
3. Administering Angiotensin 1-7. Since binding and internalization of ACE2 by SARS COV-2 presumably reduces the systemic availability of ACE2 to generate the protective molecule Ang 1-7, administration of Ang 1-7 to the patient could be a strategy to restore the vasodilatory effect of this molecule. Determining the correct dosage would be critical, in part to ensure that over-dosing does not induce a hypotensive state. Animal experiments could help determine the optimal dosing strategy and potential viability of this approach. One, presumably major, drawback of using Ang 1-7 as a treatment is that it apparently has a very short half-life in blood, on the order of a couple of minutes. This time period is roughly the time it takes for one pass of blood through the circulatory system. Therefore, the effective dosage in the body would drop rapidly and dramatically. An alternative means of carrying out this same strategy might be to dose a patient's blood with Ang 1-7 using a combinatorial apparatus similar to a modified dialysis system in which the dialysate solution contains a constant concentration of Ang 1-7 to be administered at a steady state dose in a countercurrent flow in the machine, as further described below.
4. Administering a MAS receptor agonist. Because of the short half-life of Ang 1-7 in the bloodstream, an alternative strategy would be to administer an agent that could mimic the activity of Ang 1-7 but with improved pharmacodynamic properties. One such possibility would be an agonist for the Mas receptor, which is the target at which Ang 1-7 acts to effect its vasodilatory and anti-inflammatory protective effects. A selective Mas receptor agonist, AVE0991, has been reported in the literature (Lee et al., 2015) and is the subject of numerous articles showing relevant therapeutic or protective effects in animal studies. Another Mas receptor agonist is CGEN-856S, which also has been shown to have positive effects in animal studies (Santos et al., 2018). Neither compound, however, appears to have advanced to human clinical trials.

Limitations and Adverse Outcome of Ventilator Use in COVID-19 Patients

For critical cases of COVID-19, a standard procedure in an attempt to increase oxygen flow to the patient has been to place the patient on a mechanical ventilator, usually in the critical or intensive care unit. In this procedure, tubing is inserted into the patient's airway (i.e., intubation) and attached to a device (ventilator) that forces air into the patient's lungs. This process is considered standard practice for treating severe respiratory infections, which has been the generally accepted assessment of COVID-19 based on reduced blood oxygen levels of patients, and on sections of opacity seen in lung scans. The idea is that increased oxygenation of lung tissue inside the lungs will help maintain the function of the lungs while the patient clears the respiratory infection through their immune response. This strategy presupposes that the lung infection is causing the low blood oxygenation symptom. However, if the low blood oxygen levels are due to coagulation or cardiovascular perturbations, such as (a) vasoconstriction of blood flow (from reduced ACE2 conversion of Ang II to Ang 1-7) limiting blood flow through lung capillaries or (b) lectin complement pathway mediated destruction of red blood cells and clotting leading to reduced 02 carrying capacity of blood, then it is likely that mechanical ventilation into the lungs will have a limited beneficial effect on oxygen levels throughout the body. In fact, mechanical ventilation may have a deleterious effect in the most severe cases of COVID-19 in that the increased pressure from intubation on the damaged areas of the lungs may cause more damage or rupture at those weakened areas, adding to the degree of pulmonary trauma.

In a study of 5700 COVID-19 hospitalized patients in the New York City area, Richardson et al (2020) reported that of the cohort of 2634 patients who were discharged or died at the time of the study's interim analysis, 14.2% had been admitted to the intensive care unit (ICU), 12.2% were put on mechanical ventilators, and 21% had died. Mortality for those requiring mechanical ventilation was a staggering 88%. Mortality rates for those who received mechanical ventilation in the 18-to-65 and older-than-65 age groups were 76.4% and 97.2%. However, mortality rates for those in the 18-to-65 and older-than-65 age groups who did not receive mechanical ventilation were far lower at 19.8% and 26.6%. There have been similar reports from China and in the U.S. about very high mortality rates among COVID-19 patients who were placed on mechanical ventilators. Clearly, and surprisingly given the prevailing view that COVID-19 is a respiratory infection, these data suggest ventilators don't appear to be an effective treatment for COVID-19 and may even worsen outcome.

Diabetes, Obesity, Cardiovascular Disease, and Chronic Kidney Disease Represent a High Percentage of Morbidity and Mortality in COVID-19 Patients Presenting in Severe or Critical Condition

Diabetes is a chronic disease in which your body does not properly regulate the level of sugar (glucose) in your bloodstream, due to either an inability to produce sufficient insulin by the pancreas or cells in the body become resistant to taking up glucose to use as an energy source. The former cause, called type 1 diabetes (T1D), is thought to be due to an autoimmune reaction whereby the pancreas loses its ability to produce insulin. T1D is early onset and is usually diagnosed in children or young adults. The latter cause, called type 2 diabetes (T2D), results from a gradual loss of cells' ability to take up glucose and a resulting build-up of glucose in circulation. T2D incidence increases with age as a progression to more severe disease. According to the CDC, there are about 27 million diagnosed diabetics in the U.S. (about 8% of the population), of which 5-10% have T1D and the other 90-95% have T2D. In addition, another 88 million have pre-diabetes in the U.S. The prevalence of diagnosed diabetes by race is Caucasians 9.4%; Blacks 13.3%; Hispanic 10.3%; and Asian 11.2%. By age it is 18-44 years old 3.0%; 45-64 years old 13.8%; and >65 years old 21.4%. By sex it is 11.0% men and 9.5% women.

The progression of diabetes leads to a range of severe complications and to other related diseases. Diabetics have a high risk of atherosclerosis, a leading cause of peripheral artery disease. Fatty deposits build up in arteries leading to the arms and legs, narrowing and stiffening them so that blood flow becomes constricted. Diabetes is one of the leading causes of kidney (renal) disease, also known as diabetic nephropathy. Diabetes is also a main risk factor for end-stage renal disease, the most severe form of kidney disease.

Richardson et al (2020) also reported that of the 5700 hospitalized COVID-19 patients, 34% presented with a known history of diabetes, the third most common co-morbidity on entry. The second most common co-morbidity was obesity, at 42%. It is estimated that the incidence of diabetes in the United States is significantly higher than the diagnosed patient numbers, and obesity is considered a leading indicator of development of diabetes (prediabetes). Therefore, the actual percentage of those with diabetes presenting with COVID-19 in this study was probably higher than 34%. The highest presenting co-morbidity was hypertension (56%).

Of the 2634 patients in the study cohort, 81 (3.2%) required kidney replacement therapy, clearly a severe adverse outcome. The high percentage of diabetics presenting with COVID-19 suggests a likely predisposing biology that could lead to enhanced kidney damage. The adverse outcomes for diabetics with predisposing or later stage renal disease and positive for COVID-19 may also in part be due to the likelihood that their access to hemodialysis therapy during mechanical ventilation was limited since dialysis units are not common in ICUs. Furthermore, the high levels of ACE2 in kidneys and the reduction in ACE2 due to SARS COV-2 infection, with resultant potential damage to capillary beds in the kidneys, suggest a likely broader need for hemodialysis support in treating COVID-19 that just for pre-existing dialysis patients.

Description of Combinatorial Apparatus for Disease Management: Example for COVID-19

One goal of this aspect of the invention is to provide an apparatus for the regular, as needed, hemodialysis treatments for diabetic patients while undergoing hospitalization for COVID-19, especially those patients who need to be provided with supplemental oxygen that would otherwise be provided by mechanical ventilators. Another goal would be the deployment of an apparatus and methods that would provide a superior option for administering supplemental oxygen and other therapeutic support to COVID-19 patients, compared with the currently used mechanical ventilators. Therapeutic support could be in the form of supplementing the dialysate used in the combinatorial apparatus with disease-specific molecules that are able to diffuse through the dialysate tubing in the system into the patient's blood, and/or to remove deleterious or excess diffusible molecules from the blood while passaging through the apparatus. Yet another aspect of therapeutic support could be including a capture module in the combinatorial apparatus that would enable larger molecules or other substances or agents, including viral particles, that cannot pass through the dialysis tubing to be bound to an immobilized capture substrate in the module and removed from the patient's blood stream. Alternatively, reactive molecules such as enzymes, could be immobilized to surfaces in the capture module, in which case the reactive molecules would act upon molecules in the patient's blood to alter, inactivate, or otherwise modify them as part of a therapeutic regimen. The following descriptions use therapeutic intervention in COVID-19 patients as examples. It should be clear however that the combinatorial apparatus described herein, or components thereof, could be widely used for management of other diseases. Advantages of this combinatorial apparatus would include (1) the ability to administer a drug or other small molecule (that is, one diffusible through the selected dialysis tubing molecular weight cut-oft) to a patient to rapidly and continuously achieve a steady state concentration of that drug or other small molecule in the blood or serum, (2) the ability to manage the desired concentration of small molecules in the blood stream by adding them or deleting them, or increasing or decreasing their concentration, in the dialysate fluid such that by countercurrent flow of the dialysate and patient blood a desired concentration of the targeted small molecule is achieved in the blood being returned to the patient's body, (3) the ability to remove larger molecules or infectious agents from the patient's blood through exposure to an immobilized capture molecule or substance through contact between the patient's blood and the exposed surface containing the immobilized molecule, without having to directly administer (e.g., by intravenous injection) the capture molecule into the patient, thus potentially eliminating or reducing potential side effects of having the capture agent (such as an antibody) circulating freely in the bloodstream, and (4) the ability to alter molecules in the patient's bloodstream through their interaction with an immobilized molecule (such as an enzyme) or other material within the capture module, again thus potentially eliminating or reducing potential side effects of having the modifying agent (such as an enzyme) circulating freely in the bloodstream if administered directly into the patient. In each of these cases, if any adverse effects of the therapeutic interventions are seen, that intervention can be immediately terminated by altering the composition of the dialysate fluid or by bypassing the patient's blood flow so it does not flow through the capture module. Other uses of this combinatorial apparatus should be readily apparent to those ordinarily skilled in the art.

Basic Hemodialysis Machine as Core Building Block of Combinatorial Apparatus

A hemodialysis machine is well known and well established in the art. There are numerous manufacturers of such machines worldwide (such as Fresnius, Baxter, etc.). There are an estimated 7500 dialysis treatment centers in the U.S. where diabetics go, usually 3-4 times per week for 3-4 hours at a time, to receive hemodialysis treatments. Each dialysis center reportedly contains an average of 10 machines, suggesting an installed base in commercial treatment centers in the U.S. of about 75,000 hemodialysis machines. In addition, home hemodialysis has been increasingly adopted. An advantage for patients of in-home dialysis is that they can perform dialysis at convenient times, such as in the evening to allow them to maintain daytime employment. Diabetics using home hemodialysis systems can learn to perform the treatments themselves, suggesting relative ease of operation of these machines. This is in sharp contrast to mechanical ventilators to supply oxygen in a hospital setting where trained technicians are required to monitor the oxygen pulses and pressure rates to conform to the patient's lung function. Home dialysis machines represent another pool of existing equipment that could become available for COVID-19 therapy, potentially allowing for earlier intervention after infection, based on adding a supplemental oxygen supply module to these machines.

Starting this novel combinatorial apparatus with a hemodialysis machine as the base upon which to build is a major advantage for use in a broad-based, fast-moving pandemic such as COVID-19, in that these machines are already widely available and more can be easily produced based on a breadth of manufacturers.

A diagram of a basic hemodialysis system, with key components and processes (from Wikipedia, https://en.wikipedia.org/wiki/Hemodialysis#/media/File:Hemodialysis-en.svg) is shown in Drawing 6.

(See Drawing #6)

Two tubes are inserted in the patient, usually in the veins and arteries in the arm. At the site of insertion, in patients who have been undergoing regular dialysis, a fistula eventually forms that fuses the vein and artery at the site so that there is access to both vessel directions and in relatively high blood flow. In patients who have not been on dialysis, access to high blood flow can also be achieved by inserting catheters into the leg (femoral) or neck (jugular). The blood being removed for the process via the catheter tubing is monitored for arterial blood pressure and circulated through the system with a pump. Typically, an injection port is included for heparin addition to prevent clotting during the dialysis process. An additional pressure monitor is often included prior to the dialyzer column itself.

The dialyzer column is the key component of the hemodialysis system. It serves to remove unwanted metabolites and other impurities from blood that are normally removed by the kidney in non-diabetic patients, and to add back electrolytes and other beneficial molecules to the blood. This is achieved by countercurrent flow and dialysis, or the movement of substances across a gradient from higher concentration to lower concentration. Blood is being pumped through the lumen of the dialysis column, from the input side to the output side. At the same time, fresh dialysate fluid is being pumped from a reservoir through fine tubing that has a specific molecular weight cut-off for molecules to be able to diffuse through the tubing's pores. That molecular weight cut-off can be altered by the choice of dialysis tubing. That fresh dialysate is pumped through the dialysis tubing inside the column starting at the opposite end from which the patient's blood is entering. The other end of the tubing exits the column at the same side as the blood inflow. In this countercurrent configuration, the unwanted molecules in the incoming blood present in higher concentrations than in the fresh dialysate cross from the blood to inside the dialysis tubing. And the desired molecules inside the fresh dialysate at concentrations higher than the flowing blood diffuse out of the tubing into the blood. After the blood exits the dialyzer, it goes through another pressure monitor then through an air trap and air detector to make sure no air bubbles are being returned to the body in the blood. Finally, the blood re-enters the body through a second catheter in the arm or other location.

Depending on numerous factors such as flow rates, the molecular weight cut-off size of the dialysis tubing, the starting concentrations of a molecule in the fresh dialysate, the starting concentration of an unwanted molecule in the blood, etc., the system can be used to adjust levels of molecules removed from and/or re-entering the bloodstream. For a kidney hemodialysis patient, the fresh dialysate generally contains purified water, glucose, and electrolytes, and the waste dialysate mostly contains metabolic byproducts, toxins, and excess electrolytes or water.

Addition of Separate Supplemental Oxygen Source

For COVID-19 patients, combining an additional component to the hemodialysis machine to allow for providing supplemental oxygen through the system to the patient in lieu of being given mechanical ventilation through the lungs could be achieved by a number of means.

First, the fresh dialysate could be enhanced with higher levels of dissolved oxygen. The concentration or saturation of oxygen in the dialysate solution would be calibrated to achieve the desired level of oxygen in the blood at the point at which the blood is leaving the dialysis column. An oxygen saturation sensor and monitor can be added to the system between the column and the point of re-entry of the blood into the patient to ensure proper oxygenation or flag levels that are too high or too low. At the same time, the level of CO₂ in the fresh dialysate could be either controlled for ambient levels consistent with desired CO₂ levels in the blood or reduced to below ambient in the fresh dialysate. In either case, the blood coming out of the patient and entering the column opposite the fresh dialysate in-flow could have higher concentrations of CO₂ that would diffuse out of the blood through the walls of the dialysis tubing into the waste dialysate until a proper equilibrium is reached. This system should allow for the simultaneous restoration of desired oxygen and carbon dioxide levels in the blood returned to the patient. As a further feature, an oxygen level or saturation sensor and CO₂ level sensor with monitors should be added to the basic system on the inflow side in the vicinity of the inflow pressure monitor. This feature would allow for adjustments to the oxygen and/or CO₂ concentrations in the fresh dialysate as the continuous flow from the system contributes to tissue oxygenation and CO₂ generation in the body.

Second, if a higher level of oxygen saturation is desired in the patient than can be achieved through equilibrium dialysis in the column, an additional oxygen injection port can be added in the blood flow tubing after the dialysis column and before the air trap/air detector on the outflow side. This addition would be especially useful in combination with the inflow side oxygen sensor noted above, in the event that the equilibrium dialysis contribution of oxygen to the system is not adequate to maintain high enough oxygen levels on a complete cycle of the blood from dialysis outflow through the body and back to the dialysis inflow side. In this case, some level of super saturation of oxygen in the outflow side blood may be beneficial.

Not only would this system of standard hemodialysis combined with supplemental oxygen supply benefit late stage renal disease/advanced diabetic patients with more severe cases of COVID-19 who were already on dialysis, likely negating the need for traditional mechanical ventilation, it could be used in place of mechanical ventilators for non-dialysis COVID-19 patients as well. As noted above, there is a relatively high incidence (15%-30% of patients admitted to the ICU in one study from China) of severe kidney damage and/or kidney failure, perhaps due to blockage of capillaries in the kidneys, seen in severe COVID-19 patients. This combinatorial apparatus would allow for renal support in those patients, whether or not they were previously undergoing dialysis treatments. However, in those patients in which the oxygen carrying capacity of the blood has been reduced due to loss of red blood cells or hemoglobin, additional measures or components of the combinatorial apparatus and/or additional therapeutic interventions such as blood transfusion may be required. Furthermore, further steps to prevent clotting or dissolving clots may be necessary, which is partly addressed through heparin-coated dialysis tubing and heparin injection into the blood flow. Additional ant-clotting agents or strategies to reduce coagulation or vasoconstriction can be implemented through additional components of the combinatorial apparatus. One such possibility may be adding dissolved nitric oxide (NO) gas to the dialysate or injecting NO into the oxygen injection port or into a separate injection port for NO. NO has strong vasodilatory effects in the bloodstream.

The concept of oxygenating blood outside of the body by a machine other than a ventilator is partly embodied in Extra Corporeal Membrane Oxygenation, or ECMO. This procedure involves inserting a cannula and catheter into a vein, such as the femoral vein, to remove blood, oxygenating it outside of the body, and returning the blood to the patient, generally via a catheter into the femoral artery. ECMO is an adaptation of the heart-lung bypass machine often used in cardiac surgery. A limited number of severe COVID-19 patients were placed on ECMO machines rather than ventilators especially in China, with some success. In one study (Zeng et al., 2020), the mortality rate for severe COVID-19 patients on conventional therapy with ventilators of 59-71% was reduced to 46% for patients treated on ECMO machines instead. These data indicate that extracorporeal oxygenation using a combinatorial apparatus based on a hemodialysis machine with an oxygenation module would be beneficial for COVID-19 patients. The combinatorial apparatus described here would be superior to ECMO (see also Berlin et al., 2020 for ECMO limitations).in that it would provide for simpler operation, would allow for other critical support and interventions to treat COVID-19 other than simply attempting oxygenation, and it could enable procedures to restore the oxygen-carrying capacity of blood or red blood cells that is compromised in some COVID-19 patients. Furthermore, there are a relatively small number of ECMO machines in use worldwide, whereas the number of dialysis systems that could be adapted as a combinatorial apparatus for rapid deployment in a pandemic is far greater.

The concept of supporting renal function in an ICU setting is partly embodied in Continuous Renal Replacement Therapy (CRRT), in particular systems made by Baxter. CRRT includes the basic dialysis system plus modifications that support dialysis on a continuous basis in the ICU. However, it lacks a supplemental oxygenation component and other critical components of the combinatorial apparatus described herein. Furthermore, the number of CRRT units, especially in the United States, is relatively limited, and some of the key accessories of Baxter's CRRT machines have only been available in Europe. Nevertheless, CRRT systems have been used for treatment of severe COVID-19 patients with success in supporting renal function (Fu et al., 2020).

Modifications to Composition of Traditional Hemodialysis Fluids

In addition to the usual components in dialysate of electrolytes, glucose, and water, other desired small molecules that can diffuse through the molecular weight cut-off of the pores in the dialysis tubing can be added for therapeutic effect. In the most general sense, this can be a procedure to maintain a constant level or concentration of a drug or other small molecule in the patient's bloodstream. The drug or small molecule would be dissolved in the dialysate at the desired concentration. If the incoming blood from the patient had a concentration of that drug or small molecule that was below the desired level, by countercurrent flow in the dialysis system, the concentration of the drug or molecule leaving the dialysis tubing back to the patient should be increased to the same concentration as in the dialysate. Conversely, if the drug or molecule in the incoming blood from the patient is higher than the desired concentration, the excess would flow back into the waste dialysis fluid until the desired equilibrium concentration is reached.

As one example of the utility of such a procedure, with respect to COVID-19, a desired concentration of Angiotensin 1-7, which is a low molecular weight peptide, can be added to the dialysate. As described above, Ang 1-7 causes vasodilation but is likely present in reduced levels in some COVID-19 patients due to binding of SARS COV-2 to the ACE2 receptor, thereby causing adverse cardiovascular effects. Ang 1-7 has a short half-life in blood, so administering this peptide intravenously may not be effective. Adding Ang 1-7 at the desired concentration in the dialysate for continuous administration through dialysis should overcome this half-life limitation. At the same time, due to binding of SARS COV-2 to ACE2, levels of the peptide Ang II can build up in the blood, leading to vasoconstriction and adverse effects. The absence of Ang II in the dialysate could lead to diffusion of this small molecule out of the patient's blood into the waste dialysate. The end result could be restoration of balance between Ang 1-7 and Ang II that is otherwise dysregulated due to the reduced ACE2 from viral attachment.

Addition of Molecular Capture Module

An additional component of the disclosed combinatorial apparatus could be a molecular capture module inserted into the dialysis blood tubing prior to the dialysis column, on the inflow side, after the arterial pressure monitor and preferably, but not required, after the blood pump. Such a capture module would consist of a removable in-line cartridge, in a by-passable configuration, allowing the blood to flow through it and containing a reactive surface to which capture molecules can be immobilized. Preferably the cartridge would permit a reasonably high flow rate for the blood, combined with a high-surface-area-to-volume reactive surface to maximize the number of capture sites on the reactive surface that could make contact with the blood. Examples of basic capture technologies used by those ordinarily skilled in the art in the life sciences area in particular could be adapted to such a module. These technologies include affinity chromatography or affinity purification, membrane chromatography, immunocapture, lectin chromatography, etc.

In the case of treatment of COVID-19, one example of a capture site would be the ACE2 receptor immobilized on a surface in which the type of attachment and conformation of ACE2 would allow it to (1) catalyze its normal functional reaction in the body of converting angiotensin II to angiotensin (1-7) and/or (2) binding, with high affinity or preferably irreversibly, to SARS CoV-2, the causative virus of COVID-19, for which ACE2 is its natural receptor. In the former case, converting excess angiotensin II from the blood in the dialysis flow to angiotensin (1-7) by immobilized ACE2 should have a positive therapeutic effect for COVID-19, countering the deleterious effects from the reduction in ACE2 in the body by SARS CoV-2 binding to ACE2 leading to its internalization into the cell. With less ACE2 around, an excess of the blood constrictor molecule angiotensin II builds up since it can no longer be converted to the blood dilator Angiotensin (1-7). In the latter case, binding of SARS COV-2 to the immobilized ACE2 in the capture module would take free virus out of the blood serum and reduce viral load in the body, which again should have a positive therapeutic effect.

Another example of a capture site would be an immobilized antibody on the reactive surface in the capture module. Affinity chromatography based on capture of targeted molecules by antibodies created to specifically bind to the target with extremely high affinity and specificity is well known in the art. In the case of COVID-19, the antibody can be one specifically developed to bind the SARS CoV-2 virus. The effect, similar to the second function of immobilized ACE2 noted above, would be to reduce viral titers in the body by capturing SARS COV-2 outside of the body.

Yet another example of a capture site would be an immobilized lectin, which is a protein that binds to specific carbohydrate sites on glycosylated proteins. SARS virus binds to mannose specific lectins in particular, and SARS COV-2, with its expanded number of glycosylation sites, presumably does as well. For example, banana lectin or griffithsin or derivatives thereof could be immobilized on the surface of the capture module as a means to bind and remove SARS COV-2 from the bloodstream. The specific interactions of lectins and coronaviruses are described above.

Numerous other large molecules related to SARS COV-2 infection could be targeted for capture in such a module, as such critical deleterious molecules in COVID-19 patients are identified. Some examples may include pro-inflammatory cytokines such as interleukin-6, pro-coagulation molecules such as prothrombin/thrombin and fibrinogen/fibrin, etc.

An important feature of the capture module would be to be able to easily remove a used in-line module and replace it with another fresh module. A mechanism for bypassing blood flow past the capture module needs to be incorporated in the design so that the capture module can be replaced and so that the capture module can be shut off if the intended function of the capture module is no longer needed or causes an adverse effect that needs to be terminated. This bypass and removal design would be especially important in the case in which the SARS CoV-2 would be captured up to some capacity of the module, after which new capture capacity would need to be installed. Upon removing the old module, the captured virus would need to be disposed of. Due to the hazard of SARS COV-2, such a used module may need to be destroyed in its entirety, which would be feasible with a module that is economically manufactured. However, in cases in which one or more of the components are available in limited supply (potentially such as the amount of ACE2 receptor availability) or are extremely expensive to make, a method of eluting the captured molecule or virus may need to be incorporated into the design. Another important design feature will be to be able to determine when the capacity of the capture module is reached, in order to know when to change to a new capture module.

There are numerous advantages of using a capture module in a combinatorial apparatus to bind up and remove large deleterious molecules or virus particles or components vs. injecting a capture molecule such as an antibody against the deleterious molecule into the patient. First, the captured deleterious molecule or virus can actually be removed from the patient rather than just bound up in the patient. Second, side effects or off-target effects of the capture molecule can be more easily controlled in the combinatorial apparatus than if freely injected in the patient, since the capture molecule in the combinatorial apparatus is only exposed to a patient's blood and not the rest of the tissues in the body. Third, with less worry about potential systemic safety issues, a capture molecule strategy can be quickly deployed and tested in COVID-19 patients. In fact, in general, adoption of the combinatorial apparatus for clinical testing of large molecule capture strategies in non COVID-19 patients may help accelerate the early demonstration of efficacy of such molecules, at least in disease applications in which the molecule to be captured circulates in the bloodstream. Similarly, if the bound molecule in the capture module is an enzyme with a catalytic activity to be exploited as the mechanism of action on a blood-borne substrate, this combinatorial apparatus could also be generally used for early clinical proof of concept without systemic safety concerns from otherwise injecting the enzyme.

Addition of Sampling Port Modules in Combinatorial Apparatus

Yet another component of the combinatorial apparatus could be sampling ports and/or sampling sensors inline in the catheter near the site of blood outflow from the patient and near the site of blood inflow back to the patient. Such ports would allow samples to be taken to monitor patient status and management and permit external in vitro tests to be performed on the blood or serum or circulating cells. Similarly, sensors for specific molecules to be monitored, especially molecules associated with the desired action from the capture module or molecules associated with the low molecular weight diffusible additions to the dialysate. Incorporation of sensors could allow for activation of alarms, for example, if readings are outside of desired specifications, helping to reduce the hands-on time required for medical staff treating the patient. Sensors could also feed back to another component of the combinatorial apparatus to activate bypass mechanisms, alter oxygenation rates, or apply other automation features that could be built in.

A diagram of some key components of a combinatorial apparatus is shown in Diagram 7. (See Diagram #7)

Summary Comments on Potential Progression and Management of COVID-19

COVID-19 is not only (and maybe not even primarily) a respiratory disease but also a cardiopulmonary/renal disease and a coagulation disorder. Dose and exposure are likely critical for determining whether a mild or non-symptomatic response to SARS COV-2 occurs or whether more severe complications arise. A low, short-term exposure (above some minimum threshold) may allow most or all of the virus to be captured by nasal secretory cells, initiating an innate immune response primarily through TLR-7/8, with type 1 interferon production as an antiviral response. A high, short-term dose may overwhelm the nasal secretory capture capacity allowing virus (a) to move more deeply into the lungs if the high dose is sufficiently aerosolized, in which case it will infect the alveolar cells, or more likely (b) to pass into the digestive tract and infect the patient systemically through the small intestines. This is the point at which therapeutic intervention needs to be initiated. A low-dose, long-term exposure is also likely to overwhelm the nasal secretory capture capacity, plus have the disadvantage that the SARS COV-2 virus will have had time to disable the host type 1 interferon response. Depending on the dose of virus, if low enough, systemic infection may not occur, but it may. Individuals who are exposed to SARS COV-2 at low levels but over a long period of time, such as healthcare workers, may benefit from being administered prophylactic alpha or beta interferon, potentially as a periodic nasal spray. As another means to limit the chances of systemic infection through the intestines, an agent that binds up SARS COV-2 in the digestive tract may be beneficial, especially for low-dose, long-term virus exposures. Consuming bananas or other foods containing mannose-binding lectins may be an effective strategy in this case. Banana lectin has been shown to bind to the SARS virus with high affinity, which may prevent fusion with a host cell and block or limit infection in the intestines. A high-dose, long-term exposure to SARS COV-2 will most likely result in a systemic infection and high probability of severe adverse effects.

The current nasal swab diagnostic test may, or may not, identify SARS COV-2 infection in the low-dose, short-term exposure individuals, depending on the extent and location in the nasopharygea of the infecting virus and the location where the swab was taken. A critical diagnostic need would be for detection of early systemic infection, which as noted should be the point at which therapeutic intervention should begin. Some possibilities are a fecal diagnostic test (about 1-2 day delayed after entering via the mouth or nose), a blood diagnostic test, a bronchial lavage diagnostic test (probably impractical), or some type of biomarker. Cough, sputum, or diarrhea may be surrogate markers.

Once the SARS COV-2 virus enters the bloodstream, there may be a race in time by the body for mounting an effective immune response leading to T-cell responses and neutralizing antibodies vs. development of adverse cardiovascular, coagulation, and inflammatory complications. After entering the bloodstream, SARS COV-2 is likely recognized by the mannose-binding lectin (MBL) branch of the complement system. MBL likely binds to glycosylated residues on the SARS COV-2 Spike protein and/or nucleocapsid protein, activating MASP-2 and leading to tagging of the virus by the complement system. This initiates another innate immune response leading toward T-cell responses and neutralizing antibodies. However, if the SARS COV-2 exposure is especially high or prolonged, or if SARS COV-2 is replicating and producing more virus, more MBL is produced in an acute phase reaction and more MASP-2 is produced as well. MASP-2 and MASP-1, in addition to their role in activating downstream complement factors, can activate the final steps of the coagulation pathway by converting prothrombin to thrombin and fibrinogen to fibrin. This causes local microvascular clotting and thickening of the blood, manifested in some cases by symptoms that have been called Kawasaki Disease-like in children, or disseminated intravascular coagulation. In addition, the high levels of MBL tag other human glycosylated proteins containing abnormal mannose or N-acetylglucosamine residues, including modified hemoglobin in red blood cells. This tagging process marks these modified hemoglobin, more common in diabetics and individuals with kidney disease, for destruction by the complement system.

At the same time, SARS COV-2 binds to its cellular receptor, ACE2, in alveolar cells of the lung, kidney cells, cardiac cells, and the endothelium of blood vessels, all sites where ACE2 is expressed on cell surfaces. After binding to ACE2 and being activated by a serine protease such as TMPRSS2 to enhance cell membrane fusion, the ACE2:SARS COV-2 complex becomes internalized into these cells and SARS COV-2 starts the process of replication. With less ACE2 present in cells exposed to the bloodstream due to internalization, its normal substrate, angiotensin II (a vasoconstrictor), builds up and the normal product of the ACE2 enzymatic activity, angiotensin 1-7 (a vasodilator), decreases in the blood. The result is a localized vasoconstriction of capillaries in the regions of normal ACE2 expression, such as the lungs (alveolar cells) and kidney, as well as local vasoconstriction and inflammation on the lining of other blood vessels. Due to the constriction of capillaries, and potentially the thickening of the blood from coagulation activation by the MBL pathway, blood flow gets impeded through the capillaries in the lungs and kidney. This may lead to clots in the lungs (perhaps the ground glass opacity seen in some lung scans) and to reduced or blocked blood flow at the critical juncture in the capillary beds of the lungs where venous blood is being oxygenated to the arterial side from air in the lungs. This adverse cardiovascular event could contribute to the low oxygen levels seen in many hospitalized COVID-19 patients. In addition, the effect of MBL tagging of hemoglobin for destruction by complement processes could also contribute to the observed low oxygen levels in blood of COVID-19 patients, as well as the low red blood cell counts seen in many severe COVID-19 patients. Impeded or stopped blood flow in the capillary beds of the kidney due to this same mechanism could also account for the relatively high levels of kidney damage or failure seen in severe COVID-19 patients.

In many cases, low oxygen levels are seen in patients who had otherwise mostly normal lung scans or function. Some COVID-19 lung infection may be due to direct exposure of inhaled virus (i.e., respiratory) that gets deep into the lungs where ACE2 is expressed in alveolar cells, but systemic infection through the intestines may be more likely. In either case, assuming the low oxygen levels are due to the effect of vasoconstriction, clotting, fibrin deposition, and reduced red blood cells, putting a COVID-19 patient on a ventilator is unlikely to do much good in getting oxygen levels back up via pressurized air into the lungs—if the blood can't carry the oxygen, trying to force more in won't be effective. In fact, patients triaged to a mechanical ventilator have had extraordinarily high mortality rates, and by some reports, the longer a patient is on a respirator, the worse their chances of survival. Therefore, early diagnosis of systemic infection and early intervention to control excessive coagulation and cardiovascular dysregulation as adverse events is likely critical for reducing the mortality from COVID-19. Furthermore, an improved machine or procedure to provide supplemental oxygen to COVID-19 patients directly into the bloodstream, rather than into the lungs via a ventilator, is needed and likely preferred.

Some groups of COVID-19 patients are more susceptible to severe infection and adverse events. Some of these individuals may have predisposing genetic mutations or pre-existing diseases that put them more at risk. These groups need to be identified, and screening assays need to be deployed to identify those patients who may need extra intervention and triage to more intensive care settings early after infection. In some cases, these genetic factors or predisposing conditions may be important considerations for vaccine development to ensure that a vaccine does not induce the adverse effects seen in the cardiovascular and coagulation systems. Some examples that seem likely are the following:
1. Factor V Leiden is a genetic polymorphism affecting mostly Caucasians including children and may be a factor in observed cases of Kawasaki-like disease. Individuals with Factor V Leiden are less able to stimulate the anti-coagulant activity of normal Factor V, leading to increased risk of uncontrolled micro-clotting or emboli.
2. Sickle cell disease is a mutation in the globin genes for synthesizing hemoglobin and is over represented in those of Sub-Saharan Africa and Middle Eastern descent. This mutation causes red blood cells to be deformed and stiff, making them more difficult to pass through capillaries. This effect is accentuated in vasoconstriction.
3. Individuals with diabetes and kidney disease have increased abnormal glycosylation on key proteins and higher levels of circulating MBL, which may make them more vulnerable to the coagulation stimulation of MASP-1 and MASP-2 and complement activation by MBL/MASP-2.
4. Polymorphisms exist in the gene encoding MBL (mbl2) in a subset of the population which leads to low or no circulating MBL in the blood. Such individuals have an increased risk of respiratory infection in general, although they may be less prone to adverse MBL-driven coagulation effects.

More risk factors and genetic predispositions need to be identified.

For those COVID-19 patients who develop adverse coagulation, cardiovascular, and/or inflammatory complications, full clearance of the virus and development of immunity does not necessarily mean that the patient has fully recovered. Some of these adverse effects may take a longer period of time to fully resolve. As an example, in patients who have had a heart attack, a phenomenon called reperfusion injury may occur for some period of time after the blockage in the heart has been cleared. These factors need to be considered in disease management for COVID-19.

With respect to epidemiology and risk of SARS COV-2 spread, more attention should be paid to the potential for viral shedding and excretion through feces, which clearly seems to be occurring especially in more severe cases. Protocols need to be established and communicated to better manage relevant sanitation and protection, especially in hospital and nursing home settings. In addition, the potential for SARS COV-2 transmission through unprotected sexual intercourse needs to be fully evaluated, and communicated to the public if confirmed. ACE2 is highly expressed in the testes, and the activating enzyme TMPRSS2 is highly expressed in the prostate. TMPRSS2 purportedly plays a role in liquefying semen. Preliminary reports from China indicate the presence of virus in semen samples in some cases.

### REFERENCES CITED

The entirety of the references cited are hereby relied upon and incorporated by reference herein.
Hamming I, Timens W, Bulithuis M L C, Lely A T, Navis G J, van Goor H. (2004). Tissue distribution of ACE2 proteins, the functional receptor for SARS coronavirus. A first step in understanding SARS pathogenesis. J Pathol 2004; 203: 631-637.
Ziegler et al. (2020). SARS-CoV-2 Receptor ACE2 Is an Interferon-Stimulated Gene in Human Airway Epithelial Cells and Is Detected in Specific Cell Subsets across Tissues. Cell (2020), 181:1-20.
Chen, C; Gao, G; Xu, Y; Pu, L; Wang, Q; Wang, L; et al. (12 others) (2020). SARS-CoV-2-Positive Sputum and Feces After Conversion of Pharyngeal Samples in Patients With COVID-19. Annals of Internal Medicine; Annals.org pub Mar. 30, 2020. pp. 1-3.
Fuqua J L, Wanga V, Palmer K E. (2015). Improving the large scale purification of the HIV microbicide, griffithsin. BMC Biotechnol. 2015 Feb. 22; 15(1):12. pp. 1-10.
Alam A, Jiang L, Kittleson G A, Steadman K D, Nandi S., Fuqua J L, Palmer K E, Tuse′ D, McDonald K A (2018). Technoeconomic modeling of plant-based griffithsin manufacturing. Front Bioeng Biotechnol. 2018 Jul. 24; 6:102.
Li D, Jn M, Bao P, Zhao W, Zhang S. (2020) Clinical characteristics and results of semen tests among men with coronavirus disease 2019. JAMA Netw Open. 2020; 3(5):e208292. pp 1-3.
Lin B, Ferguson C, White J T, Wang S, Vessella R, True L D, Hood L, and Nelson P S. (1999). Prostate localized and androgen-regulated expression of the membrane-bound serine protease TMPRSS2. Cancer Research 59, 4180-4184, Sep. 1, 1999.
Zhou F, Yu T, Du R, Fan G, Liu Y, Liu, Z, Zhang J, Wang Y, Song B, Gu X, Guan L, Wei Y, Li H, Wu X, Xu J, Tu S, Zhang Y, Chen H, Cao B. (2020). Clinical course and risk factors for mortality of adult inpatients with Covid-19 in Wuhan, China: a retrospective cohort study. Lancet 2020; 395: 1054-62 Published Online Mar. 9, 2020.
Swanson, M. D., Winter, H. C., Goldstein, I. J., and Markovitz, D. M. (2010). A lectin isolated from bananas is a potent inhibitor of HIV replication. J. Biol. Chem. 285, 8646-8655.
Hopper J T S, Ambrose S, Grant O C, Krumm S A, Allison T M, Degiacomi M T, Tully M D, Pritchard L K, Ozorowski G, Ward A B, Crispin M, Doores K J, Woods R J, Benesch J L P, Robinson C V, Struwe W B. (2017). The Tetrameric Plant Lectin BanLec Neutralizes HIV through Bidentate Binding to Specific Viral Glycans. Structure. 2017 May 2; 25 (5):773-782.e5.
Covés-Datson E M, Dyall J, DeWald L E, King S R, Dube D, Legendre M, Nelson E, Drews K C, Gross R, Gerhardt D M, Torzewski L, Postnikova E, Liang J Y, Ban B, Shetty J, Hensley L E, Jahrling P B, Olinger G G Jr, White J M, Markovitz D M. (2019). Inhibition of Ebola Virus by a Molecularly Engineered Banana Lectin. PLoS Negl Trop Dis. 2019 Jul. 29; 13 (7):e0007595.
KEYAERTS, E; VIJGEN, L; PANNECOUQUE, C; Van Damme, E; PEUMANS, W; EGBERINK, H; BALZARINI, J: and VAN RANST, M. (2007). Plant lectins are potent inhibitors of coronaviruses by interfering with two targets in the viral replication cycle. (2007) ANTIVIRAL RESEARCH. 75(3). p. 179-187.
Li S W, Wang C Y, Jou Y J, Huang S H, Hsiao L H, Wan L, Lin Y J, Kung S H, Lin C W. (2016). SARS Coronavirus Papain-Like Protease Inhibits the TLR7 Signaling Pathway through Removing Lys63-Linked Polyubiquitination of TRAF3 and TRAF6. Int J Mol Sci. 2016 May 5; 17(5):678.
Hu Y, Li W, Gao T, Cui Y, Jin Y, Li P, Ma Q, Liu X, Cao C. (2017). The severe respiratory syndrome coronavirus nucleocapsid inhibits Type I interferon production by interfering with TRIM25-mediated RIG-I ubiquitination. J Virol. 2017 Mar. 29; 91(8):e02143-16.
Noris M, and Remuzzi G. (2013). Overview of complement activation and regulation. Semin Nephrol. 2013 November; 33(6):479-92.
Garcia-Laorden M I, Sole-Violan J, Rodriguez de Castro F, Aspa J, Briones M L, Garcia-Saavedra A, Rajas O, Blanquer J, Caballero-Hidalgo A, Marcos-Ramos J A, Hemandez-Lopez J, Rodriguez-Gallego C. (2008). Mannose-binding lectin and mannose-binding lectin-associated serine protease 2 in susceptibility, severity, and outcome of pneumonia in adults. J Allergy Clin Immunol. 2008 August; 122(2):368-74, 374.el-2.
Ip W K E, Chan K H, Law H K W, Tso G H W, Kong E K P, Wong W H S, To Y F, Yung R W H, Chow E Y, Au K L, Chan E Y T, Lim W, Jensenius J C, Turner M W, Pereis J S M, and Lau Y L. (2005). Mannose-binding lectin in severe acute respiratory syndrome coronavirus infection. J Infectious Disease. 2005 May 15; 191(10):1697-704. Epub 2005 Apr. 11.
Jenny L, Ajjan R, King R, Thiel S, Schroeder V. (2015). Plasma levels of mannan-binding lectin-associated serine proteases MASP-1 and MASP-2 are elevated in type 1 diabetes and correlate with glycaemic control. Clin Exp Immunol. 2015 May; 180(2):227-32
Richardson S, MD, MPH, Hirsch J S, MD, MA, MSB, Narasimhan M, D O, Crawford J M, MD, PhD, Mcginn T, MD, MPH, Davidson K W, PhD, MASc, and the Northwell COVID-19 Research Consortium. (2020). Presenting characteristics, comorbidities, and outcomes among 5700 patients hospitalized with COVID-19 in the New York City area. JAMA. 2020 Apr. 22: e206775.
Krarup A, Wallis R, Presanis J S, Gál P, Sim R B. (2007). Simultaneous activation of complement and coagulation by MBL-associated serine protease 2. PLoS One. 2007 Jul. 18; 2(7):e623.
Gulla K C, Gupta K, Krarup A, Gal P, Schwaeble W J, Sim R B, O'Connor C D, Hajela K. (2010). Activation of mannan-binding lectin-associated serine proteases leads to generation of a fibrin clot. Immunology. 2010 April; 129 (4):482-95.
Liang R A, Heiland I I, Ueland T, Aukrust P, Snir O, Hindberg K, Braekkan S K, Garred P, Mollnes T E, Hansen J B. (2019). Plasma levels of mannose-binding lectin and future risk of venous thromboembolism. J Thromb Haemost. 2019 October; 17 (10):1661-1669.
Jenny L, Dobó J, Gál P, Schroeder V (2015). MASP-1 of the complement system promotes clotting via prothrombin activation. Mol Immunol. 2015 June; 65(2):398-405.
Jenny L, Dobó J, Gál P, Schroeder V (2015). MASP-1 Induced Clotting—The First Model of Prothrombin Activation by MASP-1. PLoS ONE 10 (12): e0144633.
Jenny L, Noser D, Larsen J B, Dobó J, Gál P, Pál G, Schroeder V. (2019). MASP-1 of the complement system alters fibrinolytic behaviour of blood clots. Mol Immunol. 2019 October; 114:1-9.
Jordan J. E., Montalto M. C., Stahl G. L. (2001). Inhibition of mannose-binding lectin reduces postischemic myocardial reperfusion injury. Circulation. 2001; 104:1413-1418.
Pavlov, V I; Tan, Y S; McClure, E E; La Bonte, L R; Zou, C; Gorsuch, W B; and Stahl, G L. (2015). Human Mannose-Binding Lectin Inhibitor Prevents Myocardial Injury and Arterial Thrombogenesis in a Novel Animal Model. American Journal of Pathology, Vol. 185, No. 2, February 2015.
Zhou P, Tachedjian M, Wynne J W, Boyd V, Cui J, Smith I, Cowled C, Ng J H J, Mok L, Michalski W P, Mendenhall I H, Tachedjian G, Wang L-F and Baker M. (2015) Contraction of the type I IFN locus and unusual constitutive expression of IFN-alpha in bats. 2696-2701 PNAS, Mar. 8, 2015, vol. 113, no. 10.
Guillon, P; Clément, M; Sébille, V; Rivain, J; Chou, C; Ruvoën-Clouet, N; Le Pendu, J. (2008). Inhibition of the interaction between the SARS-CoV spike protein and its cellular receptor by anti-histo-blood group antibodies. Glycobiology 2008 December; 18(12):1085-93.
O'Keefe B R, Giomarelli B, Barnard D L, Shenoy S R, Chan P K S, McMahon J B, Palmer K E, Barnett B W, Meyerholz D K, Wolford-Lenane C L, McCray P B (2010). Broad-spectrum in vitro activity and in vivo efficacy of the antiviral protein griffithsin against emerging viruses of the family Coronaviridae. J. Virol. 2010 March, 84(5):2511-21.
Micewicz E D, Cole A L, Jung C-L, Luong H, Phillips M L, Pratikhya P, Sharma S, Waring A J, Cole A M, Ruchala P. (2010). Grifonin-1: a small HIV-1 entry inhibitor derived from the algal lectin, Griffithsin. PLoS ONE. 2010 Dec. 16; 5(12):e14360.
Covés-Datson E M, Dyall J, DeWald L E, King S R, Dube D, Legendre M, Nelson E, Drews K C, Gross R, Gerhardt D M, Torzewski L, Postnikova E, Liang J Y, Ban B, Shetty J, Hensley L E, Jahrling P B, Olinger G G Jr, White J M, Markovitz D M. (2019). Inhibition of Ebola Virus by a Molecularly Engineered Banana Lectin. PLoS Negl Trop Dis. 2019 Jul. 29; 13(7):e0007595.
Swanson M D, Boudreaux D M, Salmon L, Chugh J, Winter H C, Meagher J L, Andrd S, Murphy P V, Oscarson S, Roy R, King S, Kaplan M H, Goldstein I J, Tarbet E B, Hurst B L, Smee D F, de la Fuente C, Hoffmann H H, Xue Y, Rice C M, Schols D, Garcia J V, Stuckey J A, Gabius H J, Al-Hashimi H M, Markovitz D M. (2015). Engineering a therapeutic lectin by uncoupling mitogenicity from antiviral activity. Cell. 2015 Oct. 22; 163(3):746-58.
Michelow I C, Lear C, Scully C, Prugar L I, Longley C B, Yantosca L M, Ji X, Karpel M, Brudner M, Takahashi K, Spear G T, Ezekowitz R A, Schmidt E V, Olinger G G (2011). High-dose mannose-binding lectin therapy for Ebola virus infection. J Infect Dis. 2011 Jan. 15; 203(2):175-9.
Heja D, Harmat V, Fodor K, Wilmanns M, Dobo J, Kekesi K A, Zavodszky P, Gal P, and Pal G. (2012). Monospecific Inhibitors Show That Both Mannan-binding Lectin-associated Serine Protease-1 (MASP-1) and -2 Are Essential for Lectin Pathway Activation and Reveal Structural Plasticity of MASP-2. April 2012 Journal of Biological Chemistry 287(24):20290-300.
Gao, T.; Hu, M.; Zhang, X.; et al. (32 others). (2020). Highly pathogenic coronavirus N protein aggravates lung injury by MASP-2-mediated complement over-activation. MedRxiv (preprint). doi: https://doi.org/10.1101/2020.03.29.20041962.
Soler, M; Wysocki, J; and Batlle, D. (2013). ACE2 alterations in kidney disease. Nephrol Dial Transplant (2013) 28: 2687-2697.
Cheng, Y; Luo, R; Wang, K; Zhang, M; Wang, Z; Dong, L; Li, J; Yao, Y; Ge, S; and Xu, G. (2020). Kidney disease is associated with in-hospital death of patients with COVID-19. Kidney International (2020) 97, 829-838.
Nishio K, Kashiki S, Tachibana H, and Kobayashi Y. (2011). Angiotensin-converting enzyme and bradykinin gene polymorphisms and cough: A meta-analysis. World J Cardiol 2011; 3(10): 329-336.
Ferrario, C; Jessup, J; Chappell, M; Averill, D; Brosnihan, K B; Tallant, A; Diz, D; and Gallagher, P. (2005). Effect of Angiotensin-Converting Enzyme Inhibition and Angiotensin II Receptor Blockers on Cardiac Angiotensin-Converting Enzyme 2. Circulation. 2005; 111:2605-2610.
Lee S, Evans M A, Chu H X, Kim H A, Widdop R E, Drummond G R, et al. (2015) Effect of a Selective Mas Receptor Agonist in Cerebral Ischemia In Vitro and In Vivo. PLoS ONE 10(11): e0142087.
Santos R A S, Sampaio W O, Alzamora A C, Motta-Santos D, Alenina N, Bader M, Campagnole-Santos M J. The ACE2/Angiotensin-(1-7)/MAS Axis of the Renin-Angiotensin System: Focus on Angiotensin-(1-7). Physiol Rev 98: 505-553, 2018.
Richardson S, MD, MPH, Hirsch J S, MD, MA, MSB, Narasimhan M, DO, Crawford J M, MD, PhD, Mcginn T, MD, MPH, Davidson K W, PhD, MASc, and the Northwell COVID-19 Research Consortium. (2020). Presenting characteristics, comorbidities, and outcomes among 5700 patients hospitalized with COVID-19 in the New York City area. JAMA. 2020 Apr. 22: e206775.
Zeng, Y; Cai, Z; Xianyu, Y; Yang, B X; Song, T; and Yan, Q. (2020). Prognosis when using extracorporeal membrane oxygenation (ECMO) for critically ill COVID-19 patients in China: a retrospective case series. Critical Care (2020) 24:148 pp. 1-3.
Berlin. D: Gulick. R: and Martinez, F. (2020). Severe Covid-19. Published on May 15, 2020, at NEJM.org. DOI: 10.1056/NEJMcp2009575.
Fu, D; Yang, B; Xu, J; Mao, Z; Zhou, C; and Xue, C. (2020). COVID-19 Infection in a Patient with End-Stage Kidney Disease. Nephron, published online Mar. 27, 2020. DOI: 10.1159/000507261 pp. 1-3.
Wikipedia, https://en.wikipedia.org/wiki/Hemodialysis#/media/File:Hemodialysis-en.svg).

What is claimed:

1. A device, or combinatorial apparatus, comprising a component (A) that includes a connection such as a catheter to a human patient's blood system, tubing that allows a patient's blood to flow or be pumped out of the patient into a dialyser chamber, dialysis tubing inside of the dialyser chamber in which dialysate fluid flows countercurrent to the patient's blood flow, additional tubing allowing the patient's blood to flow from the dialyser chamber back to the patient via a connection such as a catheter to the patient's blood system, in-line pressure monitors, and other desirable parts, potentially including but not limited to a heparin injector port to prevent clotting on the inflow side and an air trap and air detector on the outflow side to prevent air bubbles in returning blood; plus an additional component (B) that enables supplemental oxygen to be added to the blood as the blood is passing through component (A) such that the blood flowing back into the patient has a higher level of oxygen than the blood flowing into component (A) from the patient.

2. The device of claim 1 wherein component (A) is a hemodialysis machine or modification thereof.

3. The device of claim 1 wherein component (B) provides supplemental oxygen through addition to or modification of the dialysate fluid such that the dialysate fluid contains a therapeutically effective and higher oxygen concentration or oxygen carrying capacity than the oxygen concentration or oxygen carrying capacity of the patient's blood entering the combinatorial apparatus.

4. The device of claim 1 wherein component (B) provides supplemental oxygen through an oxygen injection system connected as part of the combinatorial apparatus to the tubing carrying the patient's blood at a site located between the dialysis chamber and the connection or catheter returning blood to the patient.

5. The device of claim 4 that further includes an oxygen level monitoring system that is capable of measuring the oxygen concentration in the patient's blood flowing through the combinatorial apparatus at a site prior to the location of the oxygen injection system and controlling the output of the oxygen injection apparatus in order to achieve a safe and therapeutically desirable level of oxygen in the blood being returned to the patient.

6. The device of claim 1 wherein an additional component (C) is incorporated into the device consisting of an in-line capture module containing an immobilized molecule or agent selected for its capability to interact with a specific substance within the patient's blood flowing through the device of claim 1.

7. The device of claim 6 wherein the component (C) includes a bypass mechanism that allows the blood flow through the capture module to be shut off while still allowing blood flow through the remainder of the combinatorial apparatus to continue.

8. The device of claim 6 wherein the capture module includes an immobilized antibody selected for its capability to bind and remove specific substances from the blood.

9. The device of claim 6 wherein the capture module includes an immobilized lectin selected for its capability to bind and remove specific substances from the blood.

10. The device of claim 6 wherein the capture module includes an immobilized enzyme selected for either its capability (i) to bind and remove specific substances from the blood through the enzyme's function as a receptor for that specific substance, or (ii) to modify the structure or properties of specific substances in the blood through the enzyme's function as a catalyst of that modification reaction.

11. The device of claim 6 wherein a capture molecule in the capture module is selected to be a treatment for SARS COV-2 infection or for ameliorating adverse effects or symptoms of COVID-19.

12. The device of claim 11 wherein the capture molecule is selected from the following: an antibody that has affinity for the Spike protein of SAR COV-2; a lectin that has affinity for mannose and/or N-acetylglucosamine residues including but not limited to mannose-binding lectin (MBL), banana lectin, or griffithsin or derivatives thereof; or angiotensin converting enzyme-2 (ACE-2) or modifications thereof.

13. A method of treating a human patient for a disease, infection, adverse event, drug or vaccine side effect, or other medical condition wherein the patient is connected to the device of claim 1 such that the patient's blood flows through the combinatorial apparatus.

14. The method of claim 13 wherein the human patient is in need of supplemental oxygen as a result of a disease, infection, adverse event, drug or vaccine side effect, or other medical condition, and such supplemental oxygen is provided directly into the patient's bloodstream through the function of the device of claim 1 in lieu of supplemental oxygen being provided to the patient directly into the lungs through a mechanical ventilator.

15. The method of claim 14 wherein the disease, infection, adverse event, drug or vaccine side effect, or other medical condition is SARS COV-2 infection, COVID-19, or related thereto.

16. The method of claim 14 wherein the patient (i) has advanced kidney disease requiring hemodialysis or has developed adverse kidney function that requires treatment as a result of a disease, infection, drug or vaccine side effect, or other medical condition and (ii) at the same time requires supplemental oxygen.

17. The method of claim 13 wherein the dialysate fluid in the combinatorial device includes a therapeutically effective concentration of one or more molecules that can pass through the dialysis tubing into the patient's blood and that can correct an imbalance of that molecule in the patient's body as a means of treating a disease, infection, adverse event, drug or vaccine side effect, or other medical condition.

18. The method of claim 17 wherein the molecule included in the dialysate fluid is angiotensin 1-7 and the disease, infection, adverse event, drug or vaccine side effect, or other medical condition is SARS COV-2 infection, COVID-19, or related thereto.

19. A method of treating a human patient for a disease, infection, adverse event, drug or vaccine side effect, or other medical condition wherein the patient is connected to the device of claim 6 such that the patient's blood flows through the combinatorial apparatus.

20. The method of claim 19 wherein the disease, infection, adverse event, drug or vaccine side effect, or other medical condition is SARS COV-2 infection, COVID-19, or related thereto.

21. The method of claim 20 wherein the capture molecule is selected from the following: an antibody that has affinity for the Spike protein of SAR COV-2; a lectin that has affinity for mannose and/or N-acetylglucosamine residues including but not limited to mannose-binding lectin (MBL), banana lectin, or griffithsin or derivatives thereof; or angiotensin converting enzyme-2 (ACE-2) or modifications thereof.
